SEQUENCE LISTING (19 sequences)

SEQ ID NO: 1 - DNA, 3000 bp, Arabidopsis thaliana
  CDS (766)..(843)
taaaaaaaaatcatatcagtatttgtttattacaaggaaaaagtataaccttttacaagaaaatatactttttctcaagaaatttgcctattttgccactagaacttcagattctcaaaaaaaaaacttgtttttttgtttttctttttttttatttcaatttttcattaatataccctctatttaactgataaaggcaagttgcaaaagagggtgtgtgtttgccccactcctcacataatgtcataataatacaatcttcgaagttttcgcaattcacattccttttttccaagtcccaatacatacacatatacaaacacacacatatacacccaaacgttatgtaactgcaaaaaaaatatcgaaacgagattctacaattagtctctagtcaaagagcttcatggcaatggtgtgaccccaaagttagtcttcacatgtttcttaagtttcaaacattgaatttttttccttttataaatagttaagacttatcttcattttctaaaatagtcaagatttatattcctcttaaaaaataatttatttccttcctaagaagaattattagatttattttctttgctagtatagattgatgaaagtgaggaaatgggagaagaaatgaagaacacaggatgtgtcttcttcttctaagtcactaacaaaatcaacaaagaggagaagccattattatataatagagagattgagagaagagatttatccaaaaaaatattgcaattcttcttggagtgaataatgccagtccctcttgcaccatatccaacacctccggcaccggcactggctccgtcgtacaacactcctccggcaaatggtaaaatctttcttaaatggacaagctatttttaatccaaaaagatcctctgttttcatattcttttacaaaagttttagtttcttataagattgattttttggctttattaaacaagattgtttttggtcatgcatacttggaaaaatcaaatattttcagattcccaatttgttttactcttcatgggaaaaaattgctcaattattttcccagaaccacatttatttatttcacatgtgttcataatccatttgaatgtactttttttggttaaaaaaataaataaaaacaacttgagatttgtaggaagtacaagtgggcagagccagttagtgtgttcaggttgcagaaaccttctgatgtatcccgtcggagcaacctccgtctgttgcgccgtctgtaacgccgtcacggccgttcctccgcccggtaaaatcctttaaaccataacaaaagagtttggttaaagtcttaaagagatctttgagttttgtaataactgattgggcaatgcaggaacggagatggcacagttggtatgtggaggatgtcatacactcttaatgtacattcgtggagctacaagtgttcaatgttcttgttgtcacactgttaatctcgccctcgaaggtttacatgcatattgatctttatatattacttattaaacaaaaaacatcgatctttatatttggtgtttaatcagatttttattgactgtttttgtttgggttgtggtttagggaatatatacaagaaaccaaatccaaaatgaaaatatttttaatttttacgatcgaggtcgttatatattacaaatttattacatgttgtcgtttatcgacaatatgtattgtaaggatttaaactcagattatattgcaaactaggaaaaaatattttattatatgttctgcttagtttattatgtgtttttattcaaaaatactttttacaataaattatattgagttttggtttttatgataaaacaataaaactaggtttgtttatatatgtgtgggtctagtttagtttcgattttgagatataagataattaaaccgaattttggatttattttgcaggtcttgtttatgtttaggtttaggttgttaaaacatttcggttttaagtgtattaataactttcatgtcttgcaaatgtaatcagcaaaccaagtagcgcatgtgaattgcggaaactgcatgatgctactaatgtatcaatatggagcaagatcagtgaaatgtgccgtttgtaactttgtcacatctgttggggttagttctctttctttatccaaaaagatcggttttaaatcagtcgcaaactaacaaaattacttctcttttttttttaacatcagaacgtttctttatataatgttttatttttggttgctattgttttgataattgcttgtagggttcaacgagcacgactgattcgaagtttaacaattaaaacttggatctatctacctatcaatacatattgagttatgagcaatatagaggaagcatcaaatctttttcactctctcttcgatcaagaatccggcagtgatgagtttgaaaccattttcggaagtaaatgaaatatgtaattcgtcgaaatttctgactttggtctctttgtccgtttgtatagagctatctctcttctttggtttttgttttgttctcttaagaaaaatttgattcaccagtttgtaagagtgattcagaaatcaaagctagactttttatgcatgaaaaaaatagattgttttaaatgcttatgcatactccgatccttttcaagcattttaaagcacaaaggacgtcggtgacaattttcatttgtaagttcatcaaaatatatatgctaagaaaatttggaaatgtaaagtgaacttacaaatgaaaatacatgtaagaaacctttatacttgatagtctcaaaaataaattgttgaaccaaaaaaactataaagtattattagaatgtggtttggaatgaagagatcaataaaccttatatattagagtagaattatagatgtgggacattctaataagtatataaaccaaacaaaacttaatcgtgatacaacaaaacaatgggacgaatatgtaataaataaaacagaacccgaaaaggttttgtcc

SEQ ID NO: 2 - protein, 154 aa, Arabidopsis thaliana
MPVPLAPYPTPPAPALAPSYNTPPANGSTSGQSQLVCSGCRNLLMYPVGATSVCCAVCNAVTAVPPPGTEMAQLVCGGCHTLLMYIRGATSVQCSCCHTVNLALEANQVAHVNCGNCMMLLMYQYGARSVKCAVCNFVTSVGGSTSTTDSKFNN

SEQ ID NO: 3 - protein, 30 aa, Arabidopsis thaliana
  MISC_FEATURE (1)..(30): Xaa is any amino acid
LXCXXCRXXLMYXXGASXVXCXXCXXXXXV

SEQ ID NO: 4 - protein, 27 aa, Arabidopsis thaliana
  MISC_FEATURE (1)..(27): Xaa is any amino acid
XXCXXCXXLLMYXXGAXSVXCXXCXXV

SEQ ID NO: 5 - protein, 143 aa, Zea mays
MPVPLAPYPAPPAPFTPPNGAQSQLVCTGCRNLLMYPAGATSVCCAVCSTVTAVPAPGTEMAQLVCGGCHTLLMYIRGATSVQCSCCHTVNLAMEANQVAHVNCGNCRMLLMYQYGARSVKCAVCSFVTSVGTSPGAEQKPSS

SEQ ID NO: 6 - protein, 143 aa, Sorghum propinquum
MPVPLAPYPTPPVPFTPPNGAQSQLVCTGCRNLLMYPAGATSVCCAVCSTVTAVPAPGTEMAQLVCGGCHTLLMYIRGATSVQCSCCHTVNLAMEANQVAHVNCGNCRMLLMYQYGARSVKCAVCNFVTSVGASPGAEQKPSS

SEQ ID NO: 7 - protein, 143 aa, Oryza sativa
MPVPLAPYPTPPVPFTPPNGAQSQLVCSGCRNLLMYPAGATSVCCAVCSTVTAVPAPGTEMAQLVCGGCHTLLMYIRGATSVQCSCCHTVNLAMEANQVAHVNCGNCRMLLMYQYGARSVKCAVCNFVTSVGASPGTDQKPSS

SEQ ID NO: 8 - protein, 146 aa, Triticum aestivum
MPVPLAPYPTPPVPFTPPAPNAGAQSQLVCSGCRNLLMYPAGATSVCCAVCSTVTAVPAPGTEMAQLVCGGCHTLLMYIRGATSVQCSCCHTVNLAMEANQVAHVNCGSCRMLLMYQYGARSVKCAVCSFVTSVGASSGAEQKPSN

SEQ ID NO: 9 - protein, 154 aa, Arabidopsis thaliana; identical to SEQ ID NO: 2

SEQ ID NO: 10 - protein, 154 aa, Medicago trunculata
MPVPLAPYPTPPPAPAPAPAPYTTPPTNGAQSQLVCSGCRNLLMYPVGATSVCCAVCNAVTAVPPPGTEMAQLVCGGCHTLLMYIRGATSVQCSCCHTVNLALEANQVAHVNCGNCRMLLMYQYGARSVKCAVCNFVTSVGASASTTEPQKFST

SEQ ID NO: 11 - protein, 145 aa, Solanum tuberosum
MPVPLAPYPTPPTPFTPPANGTQSQLVCSGCRNLLLYPVGATSVCCAVCNAVTAVPPPGTEMAQLVCGGCHTLLMYIRGATSVQCSCCHTVNLAMEANQVAHVNCGNCRMLLMYQYGARSVKCAVCNFVTSVGVSTSTAEQKFNS

SEQ ID NO: 12 - protein, 145 aa, Lycopersicon esculentum
MPVPLAPYPTPPTPFTPPSNGTQSQLVCSGCRNLLLYPVGATSVCCAVCNAVTAVPPPGTEMAQLVCGGCHTLLMYIRGATSVQCSCCHTVNLAMEANQVAHVNCGNCHMLLMYQYGARSVKCAVCNFVTSVGVATSTAEQKFNS

SEQ ID NO: 13 - DNA, 27 bp, Arabidopsis thaliana
acctaacaaaaagaaaagtgtgtgagg

SEQ ID NO: 14 - DNA, 27 bp, Arabidopsis thaliana
ataataacccctactagctctaacaag

SEQ ID NO: 15 - DNA, 19 bp, Arabidopsis thaliana
ctgctactttcatccaaac

SEQ ID NO: 16 - DNA, 24 bp, Arabidopsis thaliana
tgagttatgagcaatatagaggaa

SEQ ID NO: 17 - DNA, 29 bp, Arabidopsis thaliana
cattttataataacgctgcggacatctac

SEQ ID NO: 18 - DNA, 24 bp, Arabidopsis thaliana
cgaaacgcgattctacaattagtc

SEQ ID NO: 19 - DNA, 24 bp, Arabidopsis thaliana
attcactccaagaagaattgcaat

(19) United States
(12) Patent Application Publication
(10) Pub. No.: US 2005/0055738 A1
(43) Pub. Date: Mar. 10, 2005

(54) Methods and compositions for regulation of cell death in plants

(76) Inventors: Petra Epple, Carrboro, NC (US); Jeffery L. Dangl, Chapel Hill, NC (US)

Correspondence Address: JENKINS & WILSON, PA, 3100 TOWER BLVD, SUITE 1400, DURHAM, NC, 27707, US

(21) Appl. No.: 10/489,062

(22) PCT Filed: Sep. 30, 2002
(86) PCT No.: PCT/US02/30931

(30) Foreign Application Priority Data
Sep. 28, 2001 (US) 60326534

(51) Int. Cl. A01H 1/00; C12N 15/82; C07H 21/04; C07K 14/415

(52) U.S. Cl. 800/278; 530/370; 536/23.6; 435/468

(57) ABSTRACT

The present invention comprises a novel, positive regulator of cell death in Arabidopsis. This gene, LOL1, plays a role in the positive regulation of cell death in both an lsd1 mutant poised to undergo runaway cell death and in wild type plants challenged with pathogens. In another aspect of the invention, LOL1 provides enhanced disease resistance.

### CROSS REFERENCE TO RELATED APPLICATIONS

This application is based on and claims priority to U.S. Provisional Application Ser. No. 60/326,534, filed Sep. 28, 2001, herein incorporated by reference in its entirety.

### TECHNICAL FIELD

The present invention generally relates to plant breeding and plant genetics. More particularly, the invention relates to a novel positive regulator of programmed cell death and disease resistance. The invention also relates to plants and plant lines in exhibiting disease resistance and those in which the novel positive regulator of programmed cell death is disposed. Methods of breeding and engineering such plants and plant lines are also disclosed.

| | |
|---|---|
| PCD | programmed cell death |
| HR | hypersensitive response |
| ROI | reactive oxygen intermediates |
| SA | salicylic acid |
| MAP | mitogen activated protein |
| RCD | runaway cell death |
| Tₘ | melting point |
| TMV | tobacco mosaic virus |
| MCMV | maize chlorotic mottle virus |
| AMV | alfalfa mosaic virus |

| Single-Letter | Three-Letter | |
|---|---|---|
| Code | Code | Name |
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

### Functionally Equivalent Codons

| | |
|---|---|
| Amino Acid | Codons |

| | | | |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND ART

Plant biology is replete with examples of programmed cell death (PCD), yet very little is known about the relevant control mechanisms. The most familiar form of plant PCD is the hypersensitive response (HR) associated with successful plant innate immune responses (Dangl et al., in Biochemistry and Molecular Biology of Plants (Buchanan et al., eds.) (ASPP Press, Rockville, 2000) pp. 1044-1100; Morel & Danal, (1997) Cell Death and Different. 19: 17-24; Shirasu & Schulze-Lefert, (2000) Plant Molec. Biol. 44: 371-385). Here, recognition of a pathogen leads to rapid ion fluxes, production of reactive oxygen intermediates (ROI) and nitric oxide, MAP kinase signaling, transcriptional re-programming in and around the infection site, salicylic acid (SA) biosynthesis, and cell collapse (Danal & Jones, (2001) Nature 411: 826-833; Fevs & Parker, (2000) Trends Genet. 16: 449-455; McDowell & Danul, (2000) Trends Biochem. Sci. 25: 79-82). HR is required for disease resistance in some plant-pathogen interactions (Peterhänsel et al., (1997) Plant Cell 9: 1397-1409), but could simply reflect the passing of a signal threshold leading to cell death in others (Bendahmane et al., (1999) Plant Cell 11: 781-791).

Several loss of function mutations define cell death control genes in Arabidopsis (Dietrich et al., (1994) Cell 77: 565-578; Greenbera & Ausubel, (1993) Plant J. 4: 327-342; Walbot et al., in Genetic Engineering of Plants, (Kosuge & Meredith, eds.) Plenum Publishing Co., New York, 1983, vol. 3, pp. 431-442). These mutations typically also induce disease resistance responses, suggesting that they either are true negative regulators of HR cell death or that the ectopic cell death they engender is sufficient to activate defense pathways. The genes they encode are not related, to date, to commonly defined metazoan cell death regulators (Jones & Danal, (1996) Trends Plant Science 1:114-119; Korsmeyer, (1995) Trends Genet. 11: 101-105).

Two null mutant phenotypes suggest that the Arabidopsis LSD1 gene is an important negative regulator of plant PCD (Dietrich et al., (1997) Cell 88: 685-694). First, lsd1 plants are unable to control cell death initiated by pathogen recognition or by signaling chemicals that mimic SA action (Dietrich et al., (1994) Cell 77: 565-578). lsd1 is thus poised for a runaway cell death (RCD) phenotype. It has been demonstrated that superoxide is necessary and sufficient to control this poise (Jabs et al., (1996) Science 273: 1853-1856). Second, lsd1 plants are resistant to normally virulent pathogens (Dietrich et al., (1997) Cell 88: 685-694). Thus, LSD1 is formally a negative regulator of RCD and of basal disease resistance. The deduced LSD1 protein is small (189 amino acids), contains three highly related zinc-fingers, and can function as either a transcriptional regulator or as a scaffold protein (Dietrich et al., (1997) Cell 88: 685-694).

However, characterization of programmed cell death control in plants remains a long-felt and continuing need in the art. The present invention addresses the need for further characterization of programmed cell death in plants, as well as other needs in the art.

### SUMMARY OF THE INVENTION

Disclosed herein is an isolated and purified biologically active LOL1 polypeptide that acts as a positive regulator of programmed cell death in a plant. In one embodiment, the polypeptide comprises an amino acid sequence selected from the group consisting of SEQ ID NOs: 2 and 4-12, sequences having at least 85% identity with one of SEQ ID NOs: 2 and 4-12, and fragments thereof. A chimeric polypeptide comprising an amino acid sequence selected from the group consisting of SEQ ID NOs: 2 and 4-12 is also disclosed. Optionally, a polypeptide of the present invention is in a detectably labeled form. An antibody that selectively recognizes a polypeptide of the present invention is also provided.

Also disclosed herein is an isolated and purified nucleic acid sequence encoding a LOL1 polypeptide that acts as a positive regulator of programmed cell death in a plant. In one embodiment, the nucleic acid sequence comprises a nucleic acid sequence selected from the group consisting of: (a) SEQ ID NO: 1; (b) a sequence encoding a polypeptide comprising a an amino acid sequence selected from the group consisting of SEQ ID NOs: 2 and 4-12; and (c) a nucleic acid sequence capable of hybridizing under stringent conditions to a nucleic acid sequence according to (a) or (b). The nucleic acid sequence can be a DNA sequence.

A chimeric gene comprising a nucleic acid sequence as disclosed herein operatively linked to a promoter is also provided, as is a recombinant vector comprising the chimeric gene. A host cell stably transformed with the recombinant vector is also provided, as is a plant stably transformed with the recombinant vector, along with a seed, progeny and/or part thereof. Optionally, the nucleic acid sequence is present in the genome in a copy number effective to confer expression in the plant of a LOL1 polypeptide that acts as a positive regulator of programmed cell death in a plant. Also provided is a method of increasing LOL1 gene expression in a plant, where the method comprises transforming the plant with the recombinant vector. Also provided is a method of enhancing disease resistance in a plant, where the method comprises transforming a plant with the recombinant vector.

Optionally, the nucleic acid sequence is expressed in the plant at higher levels than in a wild type plant. Also optionally, the plant is selected from the group consisting of Arabidopsis, rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

A method of detecting a nucleic acid molecule that encodes a LOL1 polypeptide that acts as a positive regulator of programmed cell death in a plant in a biological sample containing nucleic acid material is also provided. In one embodiment, the method comprises: (a) hybridizing a nucleic acid sequence as disclosed herein to nucleic acid material of a biological sample under stringent hybridization conditions, to form a hybridization duplex; and (b) detecting the hybridization duplex. Optionally, the detected nucleic acid molecule further comprises a chromosome.

A method of identifying positive regulation of programmed cell death in a plant is also provided. In one embodiment, the method comprises: (a) contacting a query nucleic acid sequence derived from a plant with a probe comprising a nucleic acid sequence as disclosed herein; and (b) detecting the formation of a hybridized structure comprising the probe and the query nucleic acid sequence, the presence of a hybridized structure being indicative of positive regulation of programmed cell death in the plant.

An assay kit for detecting the presence of a LOL1 polypeptide that acts as a positive regulator of programmed cell death in a plant is also disclosed. In one embodiment, the assay kit comprises a first container containing a nucleic acid probe comprising a sequence of ten or more contiguous nucleotide bases corresponding to a fragment of a nucleic acid sequence as disclosed herein. Optionally, the kit further comprises a second container containing a detectable moiety.

Accordingly, it is an object of the present invention to provide a novel positive regulator of programmed cell death in plants. This and other objects are achieved in whole or in part by the present invention.

An object of the invention having been stated hereinabove, other objects will be evident as the description proceeds, when taken in connection with the accompanying Drawings and Examples as best described hereinbelow.

### BRIEF DESCRIPTION OF THE DRAWINGS

FIG. 1 depicts the genomic sequence and deduced amino acid sequence of the A. thaliana LOL1 gene. The amino acid sequence is shown in one-letter-code. The stop codon is indicated by an asterisk (*). A putative TATA-box is underlined. The cysteine residues of the three zinc finger domains are printed in bold and underlined. The position of the T-DNA insertion in the lol1-1 mutant (AFGC) is indicated by a triangle.

FIGS. 2A-2B shows the results of a comparison of the amino acid sequence of the LOL1 protein from various monocotyledonous and dicotyledonous plants.

FIG. 2A shows a comparison between the conserved zinc finger motifs of LSD1 and LOL1. Residues conserved in between three LSD1 and three LOL1 zinc fingers are marked in bold. X constitutes any amino acid.

FIG. 2B depicts the deduced LOL1 amino acid sequence of ESTs from Zea mays (SEQ ID NO: 5), Sorghum propinquum (SEQ ID NO: 6), Oryza sativa (SEQ ID NO: 7), Triticum aestivum (SEQ ID NO: 8), Arabidopsis thaliana (SEQ ID NO: 9), Medicago trunculata (SEQ ID NO: 10), Solanum tuberosum (SEQ ID NO: 11), and Lycopersicon esculentum (SEQ ID NO: 12). Identities are shaded in black. Percent protein identities to Arabidopsis LOL1 and the GenBank accession numbers (in some cases, the sequence of two ESTs were combined to generate the full length protein sequence) are given at the end of each line. M, monocotyledonous species; D, dicotyledonous species. Dashes have been added to simplify the visual depiction of the alignment.

FIGS. 3A and 3B depict the results of treating Arabidopsis plants of various genetic backgrounds with BTH.

FIG. 3A shows representative pictures of leaves taken at 7 days after inoculation with 300 μM BTH. The genotypes are indicated above the leaves and from left to right are are as follows: Ws-0; lsd1; lsd/lol1-1; lsd1/lol1-as (clone 9); lsd1/LOL1-s (clone 4); lsd1/LOL1-s (clone 5).

FIG. 3B depicts the conductivity of leaves from 48 to 102 hours after BTH treatment. The symbols represent the genotypes indicated at to the right of the graph and are as follows: lsd1/LOL1-s clone 4 (open square); lsd1 (open circle); lsd1/LOL1-s clone 5 (open triangle); lsd/lol1-1 (solid triangle); lsd1/lol1-as clone 9 (solid square); Ws-0 (solid circle).

FIGS. 4A and 4B depicts the results of inoculating Arabidopsis plants of various genetic backgrounds with Botiytis cinerea isolate A-1-3.

FIG. 4A shows representative pictures of leaves stained with lactophenol trypan blue 3 days after inoculation. The genotypes are indicated above the leaves and from left to right are as follows: Ws-0; lsd1; lsd/lol1-1; lsd1/lol1-as (clone 9); lsd1/LOL1-s (clone 4); lsd1/LOL1-s (clone 5).

FIG. 4B depicts the average lesion size as determined by measuring the extent of lactophenol trypan blue staining on each leaf with a caliper. Key: Ws-0, black bar; lsd1, hatched bar; lsd1/lol1-as, gray bars (clones 9 and 10); lsd1/LOL1-s, white bars (clones 4 and 5).

FIG. 5 is a graph of the conductivity of leaves immediately after infiltration with a 10 mM MgCl₂ solution containing Pst DC3000 (avrRpm1) at a concentration of 5×10⁷ cfu/ml. Control leaves infiltrated with 10 mM MgCl₂ did not show HR or increased conductivity. Genotypes are indicated to the right of the graph, and the symbols are as follows: LOL1-s clone 5 (solid square); LOL1-s clone 9 (solid triangle); Ws-0 (solid circle); lol1-as clone 9 (open triangle); lol1-1 (open square).

FIGS. 6A and 6B depict the results of spraying plants with P. parasitica isolate Emco5 (1×10⁴ spores/ml).

FIG. 6A shows images at 100× magnification of inoculated leaves stained with lactophenol trypan blue 5 days after inoculation. Pictures were taken with a Nikon ECLIPSE™E800 microscope with attached digital camera. Arrows indicate hyphae (Ws-0; left panel) and HR-like sites (LOL1-s clone 10; right panel).

FIG. 6B depicts susceptibility of the various plants as quantified by determining the number of spores produced on each genotype. Genotypes are indicated at the bottom of the graph, with line numbers designated for each genotype. From left to right the genotypes are Ws-0, white bar; lsd/lol1-1, lsd1/lol1-as (clone 9), gray bars; lsd1/LOL1-s, black bars (clones 3, 5, 7 and 10).

### BRIEF DESCRIPTION OF THE SEQUENCES IN THE SEQUENCE LISTING

SEQ ID NO: 1 is the genomic sequence of the Arabidopsis LOL1 gene. Depicted within the 3000 nucleotide genomic sequence is the deduced 154 amino acid sequence of the LOL1 protein, including the positions of the five coding exons.

SEQ ID NO: 2 is the deduced amino acid sequence of the Arabidopsis LOL1 protein.

SEQ ID NO: 3 depicts the zinc finger consensus sequence from the Arabidopsis LSD1 gene. In this sequence, non-conserved amino acids are denoted by “Xaa”, while leucine¹, arginine⁷, serine¹⁷, and valine³⁰ are semi-conserved.

SEQ ID NO: 4 depcits the zing finger consensus sequence from the Arabodopsis LOL1 gene. In this sequence, non-conserved amino acids are denoted by “Xaa”, while leucine⁹, serine¹⁸, and valine¹⁷ are semi-conserved.

SEQ ID NOs: 5-12 are the amino acid sequences encoded by the LOL1 gene from Zea mays (SEQ ID NO: 5), Sorghum propinquum (SEQ ID NO: 6), Oryza sativa (SEQ ID NO: 7), Triticum aestivum (SEQ ID NO: 8), Arabidopsis thaliana (SEQ ID NO: 9), Medicago trunculata (SEQ ID NO: 10), Solanum tuberosum (SEQ ID NO: 11), and Lycopersicon esculentum (SEQ ID NO: 12).

SEQ ID NOs: 13-15 are the sequences of primers that can be employed together in a polymerase chain reaction to detect the Arabidopsis lsd1 mutation disclosed in the Examples.

SEQ ID NOs: 16 and 17 are the sequences of primers that can be used together in a polymerase chain reaction to detect T-DNA insertion lol1-1 into the Arabidopsis LOL1 gene, as disclosed in the Examples.

SEQ ID NOs: 18 and 19 are primers that can be used in a reverse transcription-PCR reaction to amplify Arabidopsis LOL1 gene transcripts in order determine the relative mRNA levels of LOL1 present in the various antisense transgenic lines of the present invention, as disclosed in the Examples.

### DETAILED DESCRIPTION OF THE INVENTION

Programmed cell death control in plants is poorly understood. The present invention comprises a novel, positive regulator of cell death in plants, such as Arabidopsis. This gene, LOL1, is a homologue of a previously defined negative regulator of plant cell death called LSD1. Both encode small zinc finger proteins. The deduced LOL1 protein is remarkably conserved over 170-235 million years of evolution. LOL1 positively regulates cell death in both an lsd1 mutant poised to undergo runaway cell death and in wild type plants challenged with pathogens. Modest over-expression of LOL1 led to significantly enhanced disease resistance against a virulent pathogen. The present invention thus concerns in part the observation that this gene family plays a role in plant cell death control.

The present invention also concerns transformation vectors and processes for expressing the above-noted genes in plants. The transgenic plants thus created have, among other properties, disease resistance and positive regulation of programmed cell death.

A chimeric gene comprising a promoter active in plants operatively linked to a nucleic acid molecule encoding one of the above-noted genes is also disclosed, as is a recombinant vector comprising the chimeric gene. Host cells stably transformed with the recombinant vector are also disclosed, as is a plant stably transformed with the recombinant vector.

A method of modulating gene expression in a plant, comprising transforming the plant with the recombinant vector is disclosed. A method of modulating disease resistance in a plant, which, in one embodiment, comprises transforming the plant with the recombinant vector, is also disclosed.

A more detailed description of these and other aspects of the present invention is presented herein. Variations on these aspects of the invention, as well as modification to the described aspects of the invention will be apparent to those of ordinary skill in the art upon consideration of the present disclosure; such modifications and variations are within the scope of the present invention.

I. Definitions

Following long-standing patent law convention, the terms “a” and “an” mean “one or more” when used in this application, including the claims.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a polypeptide is produced from RNA.

As used herein, the term “hybridization” means the binding of a probe molecule, a molecule to which a detectable moiety has been bound, to a target sample.

As used herein, the term “sequencing” means determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, preferably at least 70%, more preferably 80% and most preferably 90% to 99% free of the materials and compounds with which is it associated in nature.

As used herein, the term “primer” means a sequence comprising, for example, two or more deoxyribonucleotides or ribonucleotides, more than three deoxyribonucleotides or ribonucleotides, more than eight deoxyribonucleotides or ribonucleotides or at least about 20 deoxyribonucleotides or ribonucleotides of an exonic or intronic region. Such oligonucleotides can be, for example, between ten and thirty bases in length.

As used herein, the term “DNA segment” means a DNA molecule that has been isolated free of total genomic DNA of a particular species. In one embodiment, a DNA segment encoding a LOL1 polypeptide refers to a DNA segment that comprises SEQ ID NO: 1, but can optionally comprise fewer or additional nucleic acids, yet is isolated away from, or purified free from, total genomic DNA of a source species, such as Arabisopsis. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains either or both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest can be dependent, inter alia, upon the specific nature of the enhancer-promoter.

As used herein, the term “biological activity” means any observable effect flowing from a LOL1 polypeptide. Representative, but non-limiting, examples of biological activity in the context of the present invention include disease resistance and modulation of PCD.

As used herein, the term “modified” means an alteration from an entity's normally occurring state. An entity can be modified by removing discrete chemical units or by adding discrete chemical units. The term “modified” encompasses detectable labels as well as those entities added as aids in purification.

As used herein, the term “LOL1” means nucleic acids encoding a functional LOL1 polypeptide. The term “LOL1” includes homologs.

As used herein, the terms “LOL1 gene product”, “LOL1 protein”, “LOL1 polypeptide”, and “LOL1 peptide” are used interchangeably and mean peptides having amino acid sequences which are substantially identical to native amino acid sequences from an organism of interest and which are biologically active in that they comprise all or a part of the amino acid sequence of a LOL1 polypeptide, or cross-react with antibodies raised against a LOL1 polypeptide, or retain all or some of the biological activity (e.g., regulation of cell death and/or disease resistance) of the native amino acid sequence or protein. Such biological activity can include immunogenicity.

As used herein, the terms “LOL1 gene product”, “LOL1 protein”, “LOL1 polypeptide”, and “LOL1 peptide” also include analogs of a LOL1 polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct LOL1 analogs. There is no need for a “LOL1 gene product”, “LOL1 protein”, “LOL1 polypeptide”, or “LOL1 peptide” to comprise all or substantially all of the amino acid sequence of a LOL1 polypeptide gene product. Shorter or longer sequences are anticipated to be of use in the invention; shorter sequences are herein referred to as “segments”. Thus, the terms “LOL1 gene product”, “LOL1 protein”, “LOL1 polypeptide”, and “LOL1 peptide” also include fusion, chimeric or recombinant LOL1 polypeptides and proteins comprising sequences of the present invention. Methods of preparing such proteins are disclosed herein and are known in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the terms “LOL1 gene” and “recombinant LOL1 gene” mean a nucleic acid molecule comprising an open reading frame encoding a LOL1 polypeptide of the present invention, including both exon and (optionally) intron sequences.

The term “gene” refers broadly to any segment of DNA associated with a biological function. A gene encompasses sequences including but not limited to a coding sequence, a promoter region, a cis-regulatory sequence, a non-expressed DNA segment is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As used herein, the term “DNA sequence encoding a LOL1 polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “intron” means a DNA sequence present in a given gene that is not translated into protein.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “chimeric protein” or “fusion protein’ are used interchangeably and mean a fusion of a first amino acid sequence encoding a LOL1 polypeptide with a second amino acid sequence defining a polypeptide domain foreign to, and not homologous with, a LOL1 polypeptide. A chimeric protein can present a foreign domain that is found in an organism that also expresses the first protein, or it can be an “interspecies” or “intergenic” fusion of protein structures expressed by different kinds of organisms. In general, a fusion protein can be represented by the general formula X-LOL1-Y, wherein LOL1 represents a portion of the protein which is derived from a LOL1 polypeptide, and X and Y are independently absent or represent amino acid sequences which are not related to a LOL1 sequence in an organism, which includes naturally occurring mutants. Analogously, the term “chimeric gene” refers to a nucleic acid construct that encodes a “chimeric protein” or “fusion protein” as defined herein.

As used herein, the term “recombination” and grammatical derivations thereof, means a re-assortment of genes or characters in combinations different from what they were in the parents, in the case of linked genes by crossing over.

As used herein, the term “plant” means an entire plant. The term “part of a plant” means the individual parts of a plant, including but not limited to seeds, leaves, stems and roots, as well as plant tissue cultures.

As used herein the term “complementary” means a nucleic acid sequence that is capable of base-pairing according to the standard Watson-Crick complementarity rules. That is, that the larger purines will always base pair with the smaller pyrimidines to form only combinations of Guanine paired with Cytosine (G:C) and Adenine paired with either Thymine (A:T) in the case of DNA or Adenine paired with Uracil (A:U) in the case of RNA.

As used herein, the term “hybridization techniques” refers to molecular biological techniques that involve the binding or hybridization of a probe to complementary sequences in a polynucleotide. Included among these techniques are northern blot analysis, Southern blot analysis, nuclease protection assay, etc.

As used herein, the terms “hybridization” and “binding” are used interchangeably in the context of probes and denatured DNA. Probes that are hybridized or bound to denatured DNA are aggregated to complementary sequences in the polynucleotide. Whether or not a particular probe remains aggregated with the polynucleotide depends on the degree of complementarity, the length of the probe, and the stringency of the binding conditions. The higher the stringency, the higher must be the degree of complementarity and/or the longer the probe.

As used herein, the term “probe” refers to an oligonucleotide or short fragment of DNA designed, known or suspected to be sufficiently complementary to a sequence in a denatured nucleic acid to be probed and to be bound under selected stringency conditions.

As used herein, the term “cloning” means separation and/or isolation of genes.

As used herein, the term “stringent hybridization conditions”, in the context of nucleic acid hybridization experiments such as Southern and Northern blot analysis, means a set of conditions under which single stranded nucleic acid sequences are unlikely to hybridize to one another unless there is substantial complementarity between the sequences. Stringent hybridization conditions can be both sequence- and environment-dependent. Longer sequences hybridize specifically at higher temperatures. An extensive guide to the hybridization of nucleic acids is found in Tijssen, (1993) Laboratory Techniques in Biochemistry and Molecular Biology-Hybridization with Nucleic Acid Probes, part 1, chapter 2, Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.15 M NaCl at 65° C. An example of stringent wash conditions is 15 minutes in 0.2×SSC buffer at 65° C. (See Sambrook (1989) for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0-8.3, and the temperature is typically at least about 30° C. Stringent conditions can also be achieved with the addition of destabilizing agents such as formamide. In general, a signal to noise ratio of 2-fold (or higher) than that observed for an unrelated probe in a particular hybridization assay indicates the presence of a specific hybridization.

The following are examples of hybridization and wash conditions that can be used to clone homologous nucleotide sequences that are substantially identical to reference nucleotide sequences of the present invention: a probe nucleotide sequence preferably hybridizes to a target nucleotide sequence in 7% sodium dodecyl sulfate (SDS), 0.5 M NaPO₄, 1 mM EDTA at 50° C. followed by washing in 2×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 1 ×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.5×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 50° C.; more preferably, a probe and target sequence hybridize in 7% sodium dodecyl sulfate (SDS), 0.5 M NaPO₄, 1 mM EDTA at 50° C. followed by washing in 0.1×SSC, 0.1% SDS at 65° C.

As used herein, the term “gene expression” means the cellular processes by which a biologically active polypeptide is produced from a DNA sequence.

As used herein, the term “vector” means a DNA molecule having sequences that enable its replication in a compatible host cell. A vector also includes nucleotide sequences to permit ligation of nucleotide sequences within the vector, wherein such nucleotide sequences are also replicated in a compatible host cell. A vector can also mediate recombinant production of an LOL1 polypeptide, as described further herein below. Some representative vectors include, but are not limited to, pBluescript (Stratagene), pUC18, pBLCAT3 (Luckow and Schutz, (1987) Nucleic Acids Res 15: 5490), pLNTK (Gorman et al., (1996) Immunity 5: 241-252), and pBAD/gIII (Stratagene). A representative host cell is an Arabidopsis cell.

II. General Considerations

Zinc finger modules are approximately 30 amino acid-long motifs found in a wide variety of transcription regulatory proteins in eukaryotic organisms. As the name implies, this nucleic acid binding protein domain is folded around a zinc ion. The zinc finger domain was first recognized in the transcription factor TFIIIA from Xenopus oocytes (Miller et al., (1985) EMBO 4:1609-1614; Brown et al., (1985) FEBS Lett. 186:271-274).

Families of zinc finger proteins often function in related processes. For example, the mammalian GATA family of transcription factors is important in erythroid and embryonic development (Charron et al., (1999) Mol. Cell Biol. 19: 4355-4365; Molkentin, (2000) J. Bio. Chem. 275: 38949-38952; Pevny et al., (1991) Nature 349: 257-260), and the mammalian IAP protein family negatively controls PCD (Deveraux & Reed, (1999) Genes Dev. 13: 239-252; Miller, (1999) Trends Cell Bio. 9: 323-328; Verhagen et al., (2001) Genome Biol. 2).

Transcriptional regulation is primarily achieved by the sequence-specific binding of proteins to DNA and RNA. Of the known protein motifs involved in the sequence specific recognition of DNA, the zinc finger protein is unique in its modular nature. To date, zinc finger proteins have been identified which contain between 2 and 37 modules. More than two hundred proteins, many of them transcription factors, have been shown to possess zinc fingers domains. Zinc fingers connect transcription factors to their target genes mainly by binding to specific sequences of DNA base pairs—the “rungs” in the DNA “ladder”.

Systemic acquired resistance (SAR) is one component of the complex system plants use to defend themselves from pathogens (Hunt & Ryals, (1996) Crit. Rev. in Plant Sci. 15, 583-606; Ryals et al., (1996) Plant Cell 8, 1809-1819). See also U.S. Pat. No. 5,614,395. SAR is an aspect of plant-pathogen responses because it is a pathogen-inducible, systemic resistance against a broad spectrum of infectious agents, including viruses, bacteria, and fungi. When the SAR signal transduction pathway is blocked, plants become more susceptible to pathogens that normally cause disease, and they also become susceptible to some infectious agents that would not normally cause disease (Gaffney et al., (1993) Science 261: 754-756; Delaney et al., (1994) Science 266: 1247-1250; Delaney et al., (1995) Proc. Natl. Acad. Sci. USA 92: 6602-6606; Delaney, (1997) Plant Phys. 113: 5-12; Bi et al., (1995) Plant J. 8: 235-245; Mauch-Mani & Slusarenko, (1996) Plant Cell 8: 203-212). These observations indicate that the SAR signal transduction pathway plays a role in maintaining plant health.

Salicylic acid (SA) accumulation appears to be required for SAR signal transduction. Plants that cannot accumulate SA due to treatment with specific inhibitors, epigenetic repression of phenylalanine ammonia-lyase, or transgenic expression of salicylate hydroxylase, which specifically degrades SA, also cannot induce either SAR gene expression or disease resistance (Gaffney et al., (1993) Science 261, 754-756; Delaney et al., (1994) Science 266, 1247-1250; Mauch-Mani & Slusarenko, (1996) Plant Cell 8: 203-212; Maher et al., (1994) Proc. Natl. Acad. Sci. USA 91: 7802-7806; Pallas et al., (1996) Plant J. 10: 281-293). Although it has been suggested that SA might serve as the systemic signal, this is currently controversial and, to date, all that is known for certain is that if SA cannot accumulate, then SAR signal transduction is blocked (Pallas et al., (1996) Plant J. 10: 281-293; Shulaey et al., (195) Plant Cell 7, 1691-1701; Vernooij et al., (1994) Plant Cell 6: 959-965).

Arabidopsis has emerged as a model system to study SAR (Uknes et al., (1992) Plant Cell 4: 645-656, incorporated by reference herein in its entirety; Uknes et al., (1993) Mol. Plant-Microbe Interact 6: 692-698; Cameron et al., (1994) Plant J. 5: 715-725; Mauch-Mani & Slusarenko, (1994) Mol. Plant-Microbe Interact 7: 378-383; Dempsey & Klessig, (1995) Bulletin de L'Institut Pasteur 93: 167-186). It has been demonstrated that SAR can be activated in Arabidopsis by both pathogens and chemicals, such as SA, 2,6-dichloroisonicotinic acid (INA) and benzo(1,2,3)thiadiazole-7-carbothioic acid S-methyl ester (BTH) (Uknes et al., (1992) Plant Cell 4: 645-656; Vernooij et al., (1995) Mol. Plant-Microbe Interact. 8: 228-234; Lawton et al., (1996) Plant J. 10: 71-82). Following treatment with either INA or BTH or pathogen infection, at least three pathogenesis-related (PR) protein genes, namely, PR-1, PR-2, and PR-5 are coordinately induced concomitant with the onset of resistance. In tobacco, the best characterized species, treatment with a pathogen or an immunization compound induces the expression of at least nine sets of genes (Ward et al., (1991) Plant Cell 3: 1085-1094). Transgenic disease-resistant plants have been created by transforming plants with various SAR genes (U.S. Pat. No. 5,614,395).

A number of Arabidopsis mutants have been isolated that have modified SAR signal transduction (Delaney, (1997) Plant Phys. 113: 5-12). The first of these mutants are the so-called lsd (lesions simulating disease) mutants and acd2 (accelerated cell death) (Dietrich et al. (1994) Cell 77: 565-577; Greenberg et al., (1994) Cell 77: 551-563). These mutants all have some degree of spontaneous necrotic lesion formation on their leaves, elevated levels of SA, mRNA accumulation for the SAR genes, and significantly enhanced disease resistance. At least seven different lsd mutants have been isolated and characterized (Dietrich et al., (1994) Cell 77: 565-577; Weymann et al., (1995) Plant Cell 7: 2013-2022).

III. Production of LOL1 Polypeptides

The native and mutated LOL1 polypeptides, and fragments thereof, of the present invention can be chemically synthesized in whole or part using techniques that are well-known in the art (see, e.g., Creighton, (1983) Proteins: Structures and Molecular Principles, W. H. Freeman & Co., New York). Alternatively, methods that are well known to those skilled in the art can be used to construct expression vectors containing a partial or the entire native or mutated LOL1 polypeptide coding sequence and appropriate transcriptional/translational control signals. These methods include in vitro recombinant DNA techniques, as described herein, synthetic techniques and in vivo recombination/genetic recombination (see, e.g., the techniques described in Sambrook et al., (1989) Molecular Cloning: A Laboratory Manual, Cold Spring Harbor Laboratory, New York, and Ausubel et al., (1992) Current Protocols in Molecular Biology John Wylie and Sons, Inc. New York, both incorporated herein in their entirety).

IV. Recombinant DNA Technology

Nucleic acids of the present invention can be cloned, synthesized, recombinantly altered, mutagenized, or combinations thereof. Standard recombinant DNA and molecular cloning techniques used to isolate nucleic acids are well known in the art. Exemplary, non-limiting methods are described by, for example, Sambrook et al., (eds.) (1989) Molecular Cloning, Cold Spring Harbor Laboratory Press, Cold Spring Harbor, New York; by Silhavy et al., (1984) Experiments with Gene Fusions, Cold Spring Harbor Laboratory Press, Cold Spring Harbor, New York; by Ausubel et al., (1992) Current Protocols in Molecular Biology John Wylie and Sons, Inc. New York; and by Glover, (ed.) (1985) DNA Cloning: A Practical Approach, MRL Press, Ltd., Oxford, U.K. Site-specific mutagenesis to create base pair changes, deletions, or small insertions are also well known in the art as exemplified by publications (see, e.g., Adelman et al., (1983) DNA 2:183; Sambrook et al., (eds.) (1989) Molecular Cloning, Cold Spring Harbor Laboratory Press, Cold Spring Harbor, New York).

Sequences disclosed or detected by methods of the invention can detected, subcloned, sequenced, and further evaluated by any measure well known in the art using any method usually applied to the detection of a specific DNA sequence including but not limited to dideoxy sequencing, PCR, oligomer restriction (Saiki et al., (1985) Bio/Technology 3: 1008-1012, allele-specific oligonucleotide (ASO) probe analysis (Conner et al., (1983) Proc. Natl. Acad. Sci. U.S.A. 80: 278), and oligonucleotide ligation assays (OLAs) (Landgren et. al., (1988) Science 241: 1007). Molecular techniques for DNA analysis have been reviewed (Landgren et. al., (1988) Science 242: 229-237).

Thus, the LOL1 genes disclosed herein can be incorporated in plant or bacterial cells using conventional recombinant DNA technology. Generally, this involves inserting DNA molecule encoding an LOL1 polypeptide as described herein into an expression system to which the DNA molecule is homologous (i.e., normally present) however a system can also be heterologous (i.e., not normally present). This insertion can be made using standard cloning procedures known in the art. A sutiable vector can contain the necessary elements for the transcription and translation of the inserted protein-coding sequences. A large number of vector systems known in the art can be used, such as plasmids, bacteriophage viruses and other modified viruses. Suitable vectors include, but are not limited to, viral vectors such as lambda vector systems λgt11, λgt10 and Charon4; plasmid vectors such as pBI121, pBR322, pACYC177, pACYC184, pAR series, pKK223-3, pUC8, pUC9, pUC18, pUC19, pLG339, pRK290, pKC37, pKC101, pCDNAII; and other similar systems.

The components of an expression system can also be modified to increase expression. For example, truncated sequences, nucleotide substitutions or other modifications can be employed. The expression systems described herein can be used to transform virtually any crop plant cell under suitable conditions. Transformed cells can be regenerated into whole plants such that the chosen form of the LOL1 gene is expressed in the transgenic plants.

IV.A. Construction of Plant Expression Cassettes

In one example, gene sequences intended for expression in transgenic plants can be assembled in expression cassettes behind a suitable promoter expressible in plants. An expression cassette can also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be transferred to the plant transformation vectors described herein. The following is a description of some components of a typical expression cassette that can be employed in the present invention.

IV.B. Promoters

The selection of the promoter used in an expression cassette can determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter can drive expression of the gene under various inducing conditions. Promoters vary in their strength, i.e., ability to promote transcription. Depending upon the host cell system utilized, any one of a number of suitable promoters can be used. The following are non-limiting examples of promoters that can be used in the expression cassettes.

IV.B.1. Constitutive Expression, the CaMV 35S Promoter

Construction of the plasmid pCGN1761 is described in the published patent application EP 0 392 225, which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker that includes NotI and XhoI sites in addition to the existing EcoRI site. This derivative is designated pCGN1761ENX. pCGN1761ENX is useful for the cloning of cDNA sequences or gene sequences (including microbial ORF sequences) within its polylinker for the purpose of their expression under the control of the 35S promoter in transgenic plants. The entire 35S promoter-gene sequence-tml terminator cassette of such a construction can be excised by HindIII, SphI, SaII, and XbaI sites 5′ to the promoter and XbaI, BamHI and BgII sites 3′ to the terminator for transfer to transformation vectors such as those described below. Furthermore, the double 35S promoter fragment can be removed by 5′ excision with HindIII, SphI, SaII, XbaI, or PstI, and 3′ excision with any of the polylinker restriction sites (EcoRI, NotI or XhoI) for replacement with another promoter.

If desired, modifications around the cloning sites can be made by the introduction of sequences that can enhance translation. This is particularly useful when overexpression of a LOL1 polypeptide is desired. For example, pCGN1761ENX can be modified by optimization of the translational initiation site as disclosed in U.S. Pat. No. 5,639,949, incorporated herein by reference.

IV.B.2.Expression Under a Chemically/Pathogen Regulatable Promoter

The double 35S promoter in pCGN1761ENX can be replaced with any other promoter of choice, which will result in suitably high expression levels. By way of example, one of the chemically regulatable promoters described in U.S. Pat. No. 5,614,395 can replace the double 35S promoter. The promoter of choice is preferably excised from its source by restriction enzymes, but can alternatively be PCR-amplified using primers that carry appropriate terminal restriction sites. Should PCR-amplification be undertaken, the promoter should be re-sequenced to check for amplification errors after the cloning of the amplified promoter in the target vector. The chemically/pathogen regulatable tobacco PR-1a promoter is cleaved from plasmid pCIB1004 (for construction, see EP 0 332 104, which is hereby incorporated by reference) and transferred to plasmid pCGN1761ENX (Uknes et al., (1992) Plant Cell 4: 645-656).

pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is done by cleavage with XhoI and blunting with T4 polymerase, followed by cleavage with HindIII and isolation of the larger vector-terminator containing fragment into which the pCIB1004 promoter fragment is cloned. This generates a pCGN1761ENX derivative with the PR-1a promoter and the tml terminator and an intervening polylinker with unique EcoRI and NotI sites. The selected coding sequence can be inserted into this vector, and the fusion products (i.e. promoter-gene-terminator) can subsequently be transferred to any selected transformation vector, including those described below. Various chemical regulators can be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395, herein incorporated by reference.

IV.B.3. Constitutive Expression, the Actin Promoter

Several isoforms of actin are known to be expressed in most cell types and consequently the actin promoter is a good choice for a constitutive promoter. In particular, the promoter from the rice ActI gene has been cloned and characterized (McElroy et al., (1990) Plant Cell 2: 163-171). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al., (1991) Mol. Gen. Genet. 231: 150-160). These incorporate the ActI-intron 1, Adhl 5′ flanking sequence and Adhl-intron 1 (from the maize alcohol dehydrogenase gene) and sequence from the CaMV 35S promoter. Vectors showing highest expression were fusions of 35S and ActI intron or the ActI 5′ flanking sequence and the ActI intron. Optimization of sequences around the initiating ATG (of the GUS reporter gene) also enhanced expression. The promoter expression cassettes described by McElroy et al. (McElroy et al., (1991) Mol. Gen. Genet. 231: 150-160) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al., (1993) Plant Cell Rep. 12: 506-509).

IV.B.4. Constitutive Expression, the Ubiguitin Promoter

Ubiguitin is another gene product known to accumulate in many cell types and its promoter has been cloned from several species for use in transgenic plants (e.g. sunflower—Binet et al., (1991) Plant Science 79: 87-94 and maize—Christensen et al., (1989) Plant Molec. Biol. 12: 619-632). The maize ubiguitin promoter has been developed in transgenic monocot systems and its sequence and vectors constructed for monocot transformation are disclosed in the patent publication EP 0 342 926 which is herein incorporated by reference. Taylor et al. (Taylor et al., (1993) Plant Cell Rep. 12: 491-495) describe a vector (pAHC25) that comprises the maize ubiguitin promoter and first intron and its high activity in cell suspensions of numerous monocotyledons when introduced via microprojectile bombardment. The ubiguitin promoter is suitable for gene expression in transgenic plants, especially monocotyledons. Suitable vectors are derivatives of pAHC25 or any of the transformation vectors described in this application, modified by the introduction of the appropriate ubiguitin promoter and/or intron sequences.

IV.B.5. Root Specific Expression

Another pattern of gene expression is root expression. A suitable root promoter is described by de Framond (de Framond, (1991) FEBS 290: 103-106) and also in the published patent application EP 0 452 269, which is herein incorporated by reference. This promoter is transferred to a suitable vector such as pCGN1761ENX for the insertion of a selected gene and subsequent transfer of the entire promoter-gene-terminator cassette to a transformation vector of interest.

IV.B.6. Wound-Inducible Promoters

Wound-inducible promoters can also be suitable for gene expression. Numerous such promoters have been described (e.g. Xu et al., (1993) Plant Molec. Biol. 22: 573-588, Logemann et al., (1989) Plant Cell 1: 151-158, Rohrmeier & Lehle, (1993) Plant Molec. Biol. 22: 783-792, Firek et al., (1993) Plant Molec. Biol. 22: 129-142, Warner et al., (1993) Plant J. 3: 191-201) and all are suitable for use with the instant invention. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA, which is wound induced and which can be used to isolate the cognate promoter using standard techniques. Similarly, Firek et al. and Warner et al. have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

IV.B.7. Pith-Preferred Expression

Patent Application WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

IV.B.8. Leaf-Specific Expression

A maize gene encoding phosphoenol carboxylase (PEPC) has been described by Hudspeth & Grula (Hudspeth & Grula, (1989) Plant Molec Biol 12: 579-589). Using standard molecular biological techniques the promoter for this gene can be used to drive the expression of any gene in a leaf-specific manner in transgenic plants.

IV.C. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons.

IV.D. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., (1987) Genes Develop. 1: 1183-1200). In the same experimental system, the intron from the maize bronze1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g., Gallie et al., (1987) Nucl. Acids Res. 15: 8693-8711; Skuzeski et al., (1990) Plant Molec. Biol. 15: 65-79).

IV.E. Targeting the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins, which is cleaved during chloroplast import to yield the mature protein (see e.g., Comai et al., (1988) J. Biol. Chem. 263: 15104-15109). These signal sequences can be fused to heterologous gene products to effect the import of heterologous products into the chloroplast (van den Broeck et al., (1985) Nature 313: 358-363). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, U.S. Pat. No. 5,639,949, herein incorporated by reference.

Other gene products are localized to other organelles such as the mitochondrion and the peroxisome (see, e.g., Unger et al., (1989) Plant Molec. Biol. 13: 411-418). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (Rogers et al., (1985) Proc. Natl. Acad. Sci. USA 82: 6512-6516).

In addition, sequences have been characterized which cause the targeting of gene products to other cell compartments. Amino terminal sequences are responsible for targeting to the ER, the apoplast, and extracellular secretion from aleurone cells (Koehler & Ho, (1990) Plant Cell 2: 769-783). Additionally, amino terminal sequences in conjunction with carboxy terminal sequences are responsible for vacuolar targeting of gene products (Shinshi et al., (1990) Plant Molec. Biol. 14: 357-368).

By fusing an appropriate targeting sequences described above to transgene sequences of interest it is possible to direct the transgene product to a given organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site that are required for cleavage. In some cases this requirement can be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described, for example, by Bartlett et al., in: Edelmann et al., (eds.) Methods in Chloroplast Molecular Biology, Elsevier pp 1081-1091 (1982) and Wasmann et al., (1986) Mol. Gen. Genet. 205: 446-453.

These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

V. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers can be preferred. Selection markers used routinely in transformation include the nptII gene, which confers resistance to kanamycin and related antibiotics (Messing & Vierra, (1982) Gene 19: 259-268; Bevan et al., (1983) Nature 304:184-187), the bar gene, which confers resistance to the herbicide phosphinothricin (White et al., (1990) Nucl. Acids Res 18: 1062, Spencer et al., (1990) Theor. Appl. Genet 79: 625-631), the hph gene, which confers resistance to the antibiotic hygromycin (Biochinger & Diggelmann, Mol Cell Biol 4: 2929-2931), and the dhfr gene, which confers resistance to methatrexate (Bourouis et al., (1983) EMBO J. 2(7): 1099-1104), and the EPSPS gene, which confers resistance to glyphosate (U.S. Pat. Nos. 4,940,935 and 5,188,642).

V.A. Transformation

Once the coding sequence of interest has been cloned into an expression system, it can then be transformed into a plant cell. Methods for transformation and regeneration of plants are well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of some representative techniques for transforming both dicotyledonous and monocotyledonous plants.

V.A.1. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., (1984) EMBO J 3: 2717-2722, Potrykus et al., (1985) Mol. Gen. Genet. 199: 169-177, Reich et al., (1986) Biotechnology 4: 1001-1004, and Klein et al., (1987) Nature 327: 70-73. In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain, which can depend of the complement of vir genes carried by the host Agrobacterium strain either on a co-resident Ti plasmid or chromosomally (e.g. strain CIB542 for pCIB200 and pCIB2001 (Uknes et al., (1993) Plant Cell 5: 159-169). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen & Willmitzer, (1988) Nucl. Acids Res. 16: 9877).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792, all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

V.A.2. Transformation of Monocotyledons

Transformation of most monocotyledon species has now also become routine. Representative techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation can have the advantage of avoiding complete vector construction and of generating transgenic plants with unlinked loci for the gene of interest and the selectable marker, enabling the removal of the selectable marker in subsequent generations, should this be regarded desirable.

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Gordon-Kamm et al., (1990) Plant Cell 2: 603-618) and Fromm et al. (Fromm et al., (1990) Biotechnology 8: 833-839) have published techniques for transformation of A188-derived maize line using particle bombardment. Furthermore, WO 93/07278 and Koziel et al. (Koziel et al., (1993) Biotechnology 11: 194-200) describe techniques for the transformation of elite inbred lines of maize by particle bombardment. This technique utilizes immature maize embryos of 1.5-2.5 mm length excised from a maize ear 14-15 days after pollination and a PDS-1000 He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al., (1988) Plant Cell Rep 7: 379-384; Shimamoto et al., (1989) Nature 338: 274-277; Datta et al., (1990) Biotechnology 8: 736-740). Both types are also routinely transformable using particle bombardment (Christou et al., (1991) Biotechnology 9: 957-962). Furthermore, WO 93/21335 describes techniques for the transformation of rice via electroporation. Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Vasil et al., (1992) Biotechnology 10: 667-674) using particle bombardment into cells of type C long-term regenerable callus, and also by Vasil et al. (Vasil et al., (1993) Biotechnology 11: 1553-1558) and Weeks et al. (Weeks et al., (1993) Plant Physiol. 102: 1077-1084) using particle bombardment of immature embryos and immature embryo-derived callus. One technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, (1962) Physiologia Plantarum 15: 473-497) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 h and are then bombarded. Twenty embryos per target plate is typical, although not critical.

An appropriate gene-carrying plasmid (such as pCIB3064 or pSG35) is precipitated onto micrometer size gold particles using standard procedures. Each plate of embryos is shot with the DuPont BIOLISTICS® helium device using a burst pressure of about 1000 psi using a standard 80 mesh screen. After bombardment, the embryos are placed back into the dark to recover for about 24 h (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

More recently, tranformation of monocotyledons using Agrobacterium has been described. See WO 94/00977 and U.S. Pat. No. 5,591,616, both of which are incorporated herein by reference.

VI. Transgenic Plants

A “transgenic plant” is one that has been genetically modified to contain and express heterologous DNA sequences, either as regulatory RNA molecules or as proteins. A transgenic plant can be genetically modified to contain and express at least one homologous or heterologous DNA sequence operably linked to and under the regulatory control of transcriptional control sequences which function in plant cells or tissue or in whole plants. As used herein, a transgenic plant also refers to progeny of the initial transgenic plant where those progeny contain and are capable of expressing the homologous or heterologous coding sequence under the regulatory control of the plant-expressible transcription control sequences described herein. Seeds containing transgenic embryos are encompassed within this definition as are cuttings and other plant materials for vegetative propagation of a transgenic plant.

When plant expression of a homologous or heterologous gene or coding sequence of interest is desired, that coding sequence is operably linked in the sense orientation to a suitable promoter and advantageously under the regulatory control of DNA sequences which quantitatively regulate transcription of a downstream sequence in plant cells or tissue or in planta, in the same orientation as the promoter, so that a sense (i.e., functional for translational expression) mRNA is produced. A transcription termination signal, for example, as polyadenylation signal, functional in a plant cell is advantageously placed downstream of an LOL1 coding sequence, and a selectable marker which can be expressed in a plant, can be covalently linked to the inducible expression unit so that after this DNA molecule is introduced into a plant cell or tissue, its presence can be selected and plant cells or tissue not so transformed will be killed or prevented from growing.

Where tissue specific expression of the plant-expressible LOL1 coding sequence is desired, the skilled artisan will choose from a number of well-known sequences to mediate that form of gene expression as disclosed herein. Environmentally regulated promoters are also well known in the art, and the skilled artisan can choose from well-known transcription regulatory sequences to achieve the desired result.

A method for providing positive regulation of cell death and/or a disease resistance characteristic to a plant is therefore disclosed. The method comprises introducing to said plant a construct comprising a nucleic acid sequence encoding an LOL1 gene product operatively linked to a promoter, wherein production of the LOL1 gene product in the plant provides positive regulation of cell death and/or a disease resistance characteristic in the plant. The construct can further comprises a vector selected from the group consisting of a plasmid vector or a viral vector. The LOL1 gene product comprises a protein having an amino acid sequence as set forth in any of SEQ ID NOs: 2 and 4-12. The nucleic acid sequence can be selected from the group including, but not limited to, (a) SEQ ID NO: 1; (b) a sequence encoding a polypeptide comprising an amino acid sequence selected from the group consisting of SEQ ID NOs: 2 and 4-12; and (c) a nucleic acid sequence capable of hybridizing under stringent conditions to a nucleic acid sequence according to (a) or (b).

In an alternative embodiment, the construct further comprises another nucleic acid molecule encoding a polypeptide that provides an additional desired characteristic to the plant. Other desired characteristics include, for example, yield, drought resistance, chemical resistance (e.g. herbicide or pesticide resistance), spoilage resistance or any or other desired characteristic as would be apparent to one of ordinary skill in the art after review of the disclosure of the present invention. Representative nucleic acids sequences are described in the following U.S. patents: U.S. Pat. No. 5,948,953 to Webb (brown rot fungus resistance); U.S. Pat No. Re. 36,449 to Lebrun et al. (herbicide resistance); U.S. Pat. No. 5,952,546 to Bedbrook et al. (delayed ripening tomato plants); and U.S. Pat. No. 5,986,173 issued Nov. 16, 1999 to Smeekens et al. (transgenic plants showing a modified fructan pattern).

Optionally, the method further comprises monitoring an insertion point for the construct in the plant genome; and providing for insertion of the construct into the plant genome at a location not associated with the resistance characteristic, the desired characteristic, or both the resistance or the desired characteristic.

Disease resistance and/or positive regulation of cell death can be conferred to a wide variety of plant cells, including those of gymnosperms, monocots, and dicots. Although the gene can be inserted into any plant cell falling within these broad classes, it can be particularly useful in crop plant cells, such as rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, carrot, squash, pumpkin, zucchini, cucumber, apple, pear, quince, melon, plum, cherry, peach, nectarine, apricot, strawberry, grape, raspberry, blackberry, pineapple, avocado, papaya, mango, banana, tobacco, tomato, sorghum and sugarcane.

VII. Plant Breeding

A high-level expression of the LOL1 gene, and mutants thereof, can be incorporated into plant lines through breeding. Breeding approaches and techniques are known in the art. See, for example, Welsh, Fundamentals of Plant Genetics and Breeding, John Wiley & Sons, New York (1981); Crop Breeding, Wood (Ed.), American Society of Agronomy, Madison, Wis. (1983); Mavo, The Theory of Plant Breeding, Second Edition, Clarendon Press, Oxford (1987); Singh, Breeding for Resistance to Diseases and Insect Pests, Springer-Verlag, New York (1986); and Wricke & Weber, Quantitative Genetics and Selection Plant Breeding, Walter de Gruyter and Co., Berlin (1986).

VIII. Design and Preparation of LOL1 Mutants

The present invention provides for the generation of LOL1 mutants. A general discussion of the design and preparation of such mutants and structural equivalents is presented hereinbelow. The following is discussion is also generally applicable to the LOL1 wild type polypeptides of the present invention in various contexts, such as but not limited to sequence identity, functional and biological equivalents, and sequence substitutions.

VIII.A. Chimeric LOL1 Polypeptides

The generation of chimeric LOL1 polypeptides is an aspect of the present invention. Such a chimeric polypeptide can comprise a LOL1 polypeptide or a portion of a LOL1, which is fused to a candidate polypeptide or a suitable region of the candidate polypeptide, for example a LOL1 expressed in a species other than Arabidopsis. Throughout the present disclosure it is intended that the term “mutant” encompass not only mutants of a LOL1 polypeptide but chimeric proteins generated using a LOL1 as well. Thus, it is intended that the following discussion of mutant LOL1 polypeptides apply mutatis mutandis to chimeric LOL1 polypeptides and to structural equivalents thereof.

In accordance with the present invention, a mutation can be directed to a particular site or combination of sites of a wild-type LOL1. For example, a residue having a location on, at or near the surface of the polypeptide can be replaced, resulting in an altered surface charge of one or more charge units, as compared to the wild-type LOL1. Alternatively, an amino acid residue in a LOL1 can be chosen for replacement based on its hydrophilic or hydrophobic characteristics.

Such mutants can be characterized by any one of several different properties as compared with the wild-type LOL1. For example, such mutants can have an altered surface charge of one or more charge units, or can have an increase in overall stability. Other mutants can have altered substrate specificity in comparison with, or a higher specific activity than, a wild-type LOL1.

LOL1 mutants of the present invention can be generated in a number of ways. For example, the wild-type sequence of a LOL1 can be mutated at those sites identified using this invention as desirable for mutation, by oligonucleotide-directed mutagenesis or other conventional methods, such as deletion. Alternatively, mutants of a LOL1 can be generated by the site-specific replacement of a particular amino acid with an unnaturally occurring amino acid. In addition, LOL1 mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

A mutation can be introduced into a DNA sequence coding for a LOL1 using synthetic oligonucleotides. These oligonucleotides contain nucleotide sequences flanking the desired mutation sites. A mutation can be generated in the full-length DNA sequence of a LOL1 or in any sequence coding for polypeptide fragments of a LOL1.

According to the present invention, a mutated LOL1 DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired LOL1 mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to a LOL1 mutant coding sequence. For expression under the direction of the control sequences, a desired DNA sequence must be operatively linked to the control sequences; that is, the sequence must have an appropriate start signal in front of the DNA sequence encoding the LOL1 mutant, and the correct reading frame to permit expression of that sequence under the control of the control sequences and production of the desired product encoded by that LOL1 sequence must be maintained.

Any of a wide variety of well-known available expression vectors can be useful in the expression of a mutated LOL1 coding sequence of this invention. These expression vectors can be used in the techniques disclosed herein above and in the Laboratory Examples and can include, for example, vectors comprising segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences.

In addition, any of a wide variety of expression control sequences—sequences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli, the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated LOL1 polypeptides according to this invention. These hosts include, for example, bacteria, such as E. coli, Bacillus and Streptomyces, fungi, such as yeasts, plant cells, insect cells, such as Sf9 and Sf21 cells, and transgenic host cells.

It should be understood that not all expression vectors and expression systems function in the same way to express mutated DNA sequences of this invention, and to produce modified LOL1 polypeptides or LOL1 mutants. Neither do all hosts function equally well with the same expression system. One of ordinary skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

When selecting an expression control sequence, a variety of factors should also be considered. These include, for example, the relative strength of the system, its controllability and its compatibility with the DNA sequence encoding a modified LOL1 polypeptide of this invention, with particular regard to the formation of potential secondary and tertiary structures.

Hosts should be selected by consideration of their compatibility with the chosen vector, the toxicity of a modified LOL1 to them, their ability to express mature products, their ability to fold proteins correctly, their fermentation requirements, the ease of purification of a modified LOL1 and safety. Within these parameters, one of skill in the art can select various vector/expression control system/host combinations that will produce useful amounts of a mutant LOL1. A mutant LOL1 produced in these systems can be purified by a variety of conventional steps and strategies, including those used to purify the wild-type LOL1.

Once a LOL1 mutation(s) has been generated in the desired location, such as a ligand binding site, the mutants can be tested for any one of several properties of interest. For example, mutants can be screened for an altered charge at physiological pH. This can be determined by measuring the mutant LOL1 isoelectric point (pI) and comparing the observed value with that of the wild-type parent. Isoelectric point can be measured by gel-electrophoresis according to the method of Wellner (Wellner, (1971) Anal. Chem. 43: 597). A mutant LOL1 polypeptide containing a replacement amino acid located at the surface of the enzyme, as provided by the structural information of this invention, can lead to an altered surface charge and an altered pI.

VIII.B. Generation of an Engineered LOL1 or LOL1 Mutant

In another aspect of the present invention, a unique LOL1 polypeptide can be generated. Such a mutant can facilitate purification and/or can facilitate the study of the biological activity of a LOL1 polypeptide.

As used herein, the terms “engineered LOL1” and “LOL1 mutant” refer to polypeptides having amino acid sequences that contain at least one mutation in the wild-type sequence. The terms also refer to LOL1 polypeptides which are capable of exerting a biological effect in that they comprise all or a part of the amino acid sequence of an LOL1 mutant polypeptide of the present invention, or cross-react with antibodies raised against a LOL1 mutant polypeptide, or retain all or some or an enhanced degree of the biological activity of the LOL1 mutant amino acid sequence or protein. Such biological activity can include disease resistance and/or postive regulation of cell death.

The terms “engineered LOL1” and “LOL1 mutant” also includes analogs of a LOL1 mutant polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct LOL1 mutant analogs. There is no need for a LOL1 mutant polypeptide to comprise all or substantially all of the amino acid sequence of SEQ ID NOs: 2 and 4-12. Shorter or longer sequences are anticipated to be of use in the invention; shorter sequences are herein referred to as “segments”. Thus, the terms “engineered LOL1” and “LOL1 mutant” also includes fusion, chimeric or recombinant LOL1 or LOL1 mutant polypeptides and proteins comprising sequences of the present invention. Methods of preparing such proteins are disclosed herein above and are known in the art.

VlII.C. Sequence Similarity and Identity

As used herein, the term “substantially similar” means that a particular sequence varies from nucleic acid sequence of SEQ ID NO: 1 or the amino acid sequence of SEQ ID NOs: 2 and 4-12 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VIII.C.1. Sequences that are Substantially Identical to a LOL1 Sequence

Nucleic acids that are substantially identical to a nucleic acid sequence of a LOL1 sequence of the present invention (including a LOL1 mutant), such as allelic variants, genetically altered versions of the gene, etc., bind to a LOL1 sequence under stringent hybridization conditions. By using probes, particularly labeled probes of DNA sequences, one can isolate homologous or related genes. The source of homologous genes can be any species.

Between various plant species, homologs can have substantial sequence similarity, i.e. at least 85%-99% sequence identity between nucleotide sequences, including at least 86%, 87%, 88%, 89%, 90%, 91%, 92%, 93%, 94%, 95%, 96%, 97%, and 98% sequence identity. Sequence similarity is calculated based on a reference sequence, which can be a subset of a larger sequence, such as a conserved motif, coding region, flanking region, etc. A reference sequence can be, for example, at least about 18 nucleotides (nt) long, or in another example, at least about 30 nucleotides long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Bio. 48: 443, as revised by Smith et al., (1981) Adv. Appl. Math. 2:482. Briefly, the GAP program defines similarity as the number of aligned symbols (i.e., nucleotides or amino acids) that are similar, divided by the total number of symbols in the shorter of the two sequences. Parameters for the GAP program can be, for example, the default parameters, which do not impose a penalty for end gaps. See, e.g., Schwartz et al., (eds.), (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, nucleic acid sequences are “substantially identical” to specific nucleic acid disclosed herein if: (a) the nucleic acid sequence is derived from coding regions of the nucleic acid sequence shown in SEQ ID NO: 1; or (b) the nucleic acid sequence is capable of hybridization with nucleic acid sequences of (a) under stringent conditions and which encode a biologically active LOL1 gene product; or (c) the nucleic acid sequences are degenerate as a result of alternative genetic code to the nucleic acid sequences defined in (a) and/or (b). Substantially identical proteins and nucleic acids can have, for example, between about 70% and 80%, or about 81% to about 90% or about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μg/ml salmon sperm DNA and 15% formamide at 68° C. For the purposes of specifying additional conditions of high stringency, conditions can comprise, for example, a salt concentration of about 200 mM and temperature of about 45° C. One example of such stringent conditions is hybridization at 4×SSC, at 65° C., followed by a washing in 0.1×SSC at 65° C. for one hour. Another example stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

VIII.C.2. Complementarity and Hybridization to a LOL1 Sequence

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and or temperatures in excess of about 45° C. Stringent salt conditions will ordinarily be less than about 1,000 mM, less than about 500 mM, or less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, New York.

VIII.D. Functional Equivalents of a LOL1 Nucleic Acid Sequence of the Present Invention

As used herein, the term “functionally equivalent codon” is used to refer to codons that encode the same amino acid, such as the ACG and AGU codons for serine. LOL1-encoding nucleic acid sequences comprising SEQ ID NO: 1, and fragments thereof, which have functionally equivalent codons, are covered by the present invention. Thus, when referring to the sequence example presented in SEQ ID NO: 1, applicants contemplate substitution of functionally equivalent codons into the sequence example of SEQ ID NO: 1. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N— or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VIII.E. Biological Equivalents

The present invention envisions and includes biological equivalents of a LOLL polypeptide of the present invention. The term “biological equivalent” refers to proteins having amino acid sequences which are substantially identical to the amino acid sequence of a LOL1 polypeptide of the present invention and which are capable of exerting a biological effect in that they are capable of modulating cell death or cross-reacting with anti-LOL1 antibodies raised against a LOL1 polypeptide (such as a mutant LOL1 polypeptide) of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of a LOL1 polypeptide of the present invention (including a LOL1 mutant) or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs: 2 and 4-12 applicants envision substitution of codons that encode biologically equivalent amino acids, as described herein, into the sequence examples of SEQ ID NOs: 2 and 4-12, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of lIe for Leu. Changes designed by man can be introduced through the application of site-directed mutagenesis techniques, e.g., to introduce improvements to the antigenicity of the protein or to test a mutant LOL1 polypeptide of the present invention in order to modulate cell death or other activity, at the molecular level.

Amino acid substitutions, such as those which might be employed in modifying a LOL1 polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of ordinary skill in the art. It is implicit in the above discussion, however, that one of skill in the art can appreciate that a radical, rather than a conservative substitution is warranted in a given situation. Non-conservative substitutions in LOL1 polypeptides (including LOL1 mutant polypeptides) of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132). It is known that certain amino acids can be substituted for other amino acids having a similar hydropathic index or score and still retain a similar biological activity. In making changes based upon the hydropathic index, the substitution of amino acids whose hydropathic indices are within, for example, ±2, ±1, or ±0.5 of the original value can also be employed.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within, for example, ±2, ±1 or within ±0.5 of the original value can be employed.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 1 and 4-12. Recombinant vectors and isolated DNA segments can therefore variously include a LOL1 polypeptide-encoding region (including a mutant LOL1 polypeptide-encoding region) itself, include coding regions bearing selected alterations or modifications in the basic coding region, or include larger polypeptides which nevertheless comprise a LOL1 polypeptide-encoding region (including a mutant LOL1 polypeptide-encoding region) or can encode biologically functional equivalent proteins or polypeptides which have variant amino acid sequences. Biological activity of a LOL1 polypeptide can be determined, for example, by assay disclosed herein.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length being a reflection of, for example, the ease of preparation and use in the intended recombinant DNA protocol. For example, nucleic acid fragments can be prepared which include a short stretch complementary to a nucleic acid sequence set forth in SEQ ID NO: 1, such as about 10 nucleotides, and which are up to 10,000 or 5,000 base pairs in length. DNA segments with total lengths of about 4,000, 3,000, 2,000, 1,000, 500, 200, 100, and about 50 base pairs in length can also be employed.

The DNA segments of the present invention encompass biologically functional equivalents of LOL1 polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of a LOL1 of the present invention in a desired activity at the molecular level. Various site-directed mutagenesis techniques are known to those of ordinary skill in the art and can be employed in the present invention.

The invention further encompasses fusion proteins and peptides wherein a wild type or a mutant LOL1 coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a LOL1 gene, as can be obtained by isolating the 5′ non-coding sequences located upstream of the coding segment or exon, for example, using recombinant cloning and/or PCR technology and/or other methods known in the art, in conjunction with the compositions disclosed herein.

As disclosed herein above, in other embodiments, certain advantages will be gained by positioning the coding DNA segment under the control of a recombinant, or heterologous, promoter. As used herein, a recombinant or heterologous promoter is a promoter that is not normally associated with a LOL1 gene in its natural environment. Such promoters can include promoters isolated from bacterial, viral, eukaryotic, or plant cells. Naturally, it will be important to employ a promoter that effectively directs the expression of the DNA segment in the cell type chosen for expression. The use of promoter and cell type combinations for protein expression is disclosed herein above and is generally known to those of skill in the art of molecular biology (see, e.g., Sambrook et al., (1989) Molecular Cloning: A Laboratory Manual, Cold Spring Harbor Laboratory, New York, incorporated herein by reference). As discussed above, the promoters employed can be constitutive or inducible and can be used under the appropriate conditions to direct high level expression of the introduced DNA segment, such as is advantageous in the large-scale production of recombinant proteins or peptides.

IX. Methods, Compositions, and Kits for Detecting a LOL1 Polypeptide or a Nucleic Acid Molecule Encoding the Same

In another aspect of the invention, a method is provided for detecting a LOL1 polypeptide using an antibody that specifically recognizes a LOL1 polypeptide, or portion thereof. In a preferred embodiment, biological samples from an experimental subject and a control subject are obtained, and LOL1 polypeptide is detected in each sample by immunochemical reaction with the antibody. More preferably, the antibody recognizes amino acids of any one of SEQ ID NOs:2 and 4-12, and is prepared according to a method of the present invention for producing such an antibody. A kit for carrying out the method is also provided.

In one embodiment, an antibody is used to screen a biological sample for the presence of a LOL1 polypeptide. A biological sample to be screened can be a biological fluid such as extracellular or intracellular fluid, or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide. In accordance with a screening assay method, a biological sample is exposed to an antibody immunoreactive with a LOL1 polypeptide whose presence is being assayed, and the formation of antibody-polypeptide complexes is detected. Techniques for detecting such antibody-antigen conjugates or complexes are well known in the art and include but are not limited to centrifugation, affinity chromatography and the like, and binding of a labeled secondary antibody to the antibody-candidate receptor complex.

The term “immunochemical reaction”, as used herein, refers to any of a variety of immunoassay formats used to detect antibodies specifically bound to a particular protein, including but not limited to competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels), western blots, precipitation reactions, agglutination assays (e.g., gel agglutination assays, hemagglutination assays), complement fixation assays, immunofluorescence assays, protein A assays, and immunoelectrophoresis assays, etc. See Harlow & Lane (1988) for a description of immunoassay formats and conditions.

The present invention also provides antibodies immunoreactive with a LOLL polypeptide. The term “antibody” indicates an immunoglobulin protein, or functional portion thereof, including a polyclonal antibody, a monoclonal antibody, a chimeric antibody, a single chain antibody, Fab fragments, and an Fab expression library.

The term “functional portion” refers to the part of the protein that binds a molecule of interest. In a preferred embodiment, an antibody of the invention is a monoclonal antibody. Techniques for preparing and characterizing antibodies are well known in the art (See, e.g., Harlow & Lane, (1988) Antibodies: A Laboratory Manual Cold Spring Harbor Laboratory Press, Cold Spring Harbor, New York). A monoclonal antibody of the present invention can be readily prepared through use of well-known techniques such as the hybridoma techniques exemplified in U.S. Pat. No 4,196,265 and the phage-displayed techniques disclosed in U.S. Pat. No. 5,260,203.

The phrase “specifically (or selectively) binds to an antibody”, or “specifically (or selectively) immunoreactive with”, when referring to a protein or peptide, refers to a binding reaction which is determinative of the presence of the protein in a heterogeneous population of proteins and other biological materials. Thus, under designated immunoassay conditions, the specified antibodies bind to a particular protein and do not show significant binding to other proteins present in the sample. Specific binding to an antibody under such conditions can require an antibody that is selected for its specificity for a particular protein. For example, antibodies raised to a protein with an amino acid sequence encoded by any of the nucleic acid sequences of the invention can be selected to obtain antibodies specifically immunoreactive with that protein and not with unrelated proteins.

The use of a molecular cloning approach to generate antibodies, particularly monoclonal antibodies, and more particularly single chain monoclonal antibodies, are also provided. The production of single chain antibodies has been described in the art. See, e.g., U.S. Pat. No. 5,260,203. For this approach, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by heavy (H) and light (L) chain combinations in a single chain, which further increases the chance of finding appropriate antibodies. Thus, an antibody of the present invention, or a “derivative” of an antibody of the present invention, pertains to a single polypeptide chain binding molecule which has binding specificity and affinity substantially similar to the binding specificity and affinity of the light and heavy chain aggregate variable region of an antibody described herein.

In another aspect of the invention, a method is provided for detecting a nucleic acid molecule that encodes a LOL1 polypeptide. According to the method, a biological sample having nucleic acid material is procured and hybridized under stringent hybridization conditions to a LOL1 polypeptide-encoding nucleic acid molecule of the present invention. Such hybridization enables a nucleic acid molecule of the biological sample and a LOL1 polypeptide encoding-nucleic acid molecule to form a detectable duplex structure. Preferably, the LOL1 polypeptide encoding-nucleic acid molecule includes some or all nucleotides of SEQ ID NO:1.

An assay kit for detecting the presence of a LOL1 polypeptide that acts as a positive regulator of programmed cell death in a plant is also disclosed. In one embodiment, the assay kit comprises a first container containing a nucleic acid probe comprising a sequence of ten or more contiguous nucleotide bases corresponding to a fragment of a nucleic acid sequence as disclosed herein. Optionally, the kit further comprises a second container containing a detectable moiety, such as a radioactive or fluorescent moiety, as would be apparent to one of ordinary skill in the art after a review of the present disclosure.

### LABORATORY EXAMPLES

The following Examples have been included to illustrate representative modes of the invention. Certain aspects of, the following Examples are described in terms of techniques and procedures found or contemplated by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

Standard recombinant DNA and molecular cloning techniques used here are well known in the art and are described by Sambrook et al., (eds.) (1989) Molecular Cloning, Cold Spring Harbor Laboratory Press, Cold Spring Harbor, New York and by Silhavy et al., (1984) Experiments with Gene Fusions, Cold Spring Harbor Laboratory, Cold Spring Harbor, New York and by Ausubel et al., (1992) Current Protocols in Molecular Biology John Wylie and Sons, Inc. New York.

### Laboratory Example 1

### Identification and Characterization of an LSD1-Related Gene, LOL1

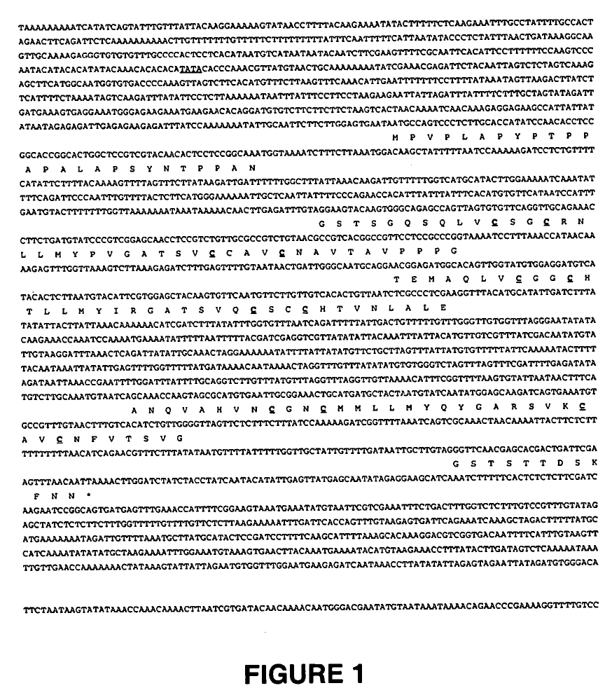

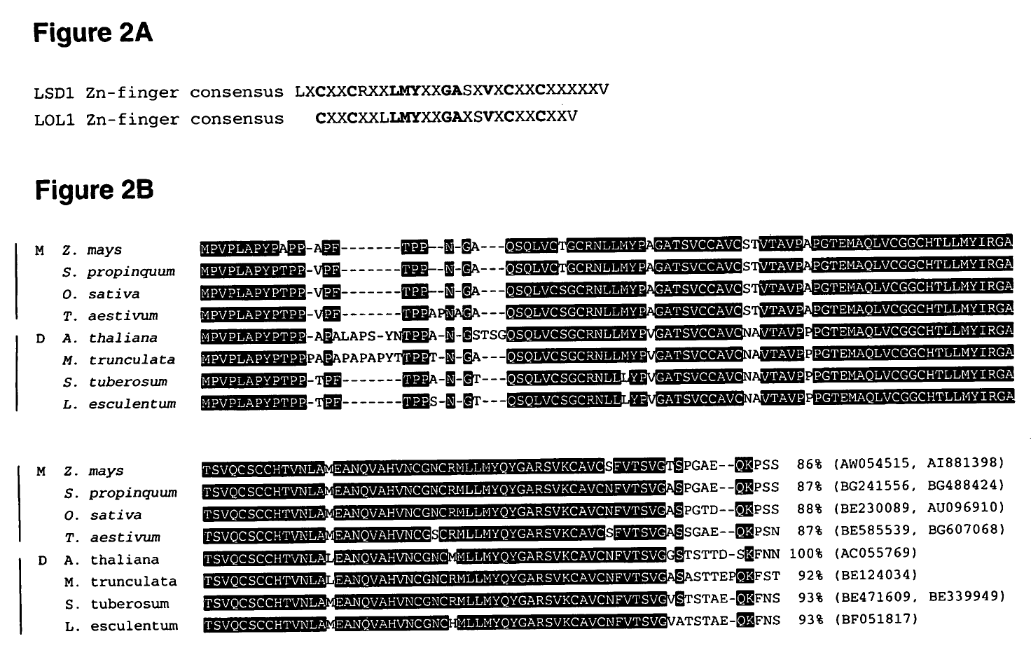

In order to identify proteins with functions similar to that of LSD1, a computer search was performed of the complete Arabidopsis genome using the internally conserved zinc finger motif sequence of LSD1 as listed in FIG. 2A: 5′-LXCXXCRXXLMYXXGASXVXCXXCXXXXXV (SEQ ID NO: 5). A small LSD1-like gene family was identified. One of these genes, LOL1, mapped to BAC clone T9G5 at the top of chromosome 1 and encodes a protein of 154 amino acids containing three LSD1-like zinc fingers (See FIG. 1). Another computer-aided homology search indicated that the nucleotide sequence of LOL1 is extremely highly conserved among monocotyledonous and dicotyledonous plants (FIG. 2B), but is absent from bacteria, yeast and animals. Like LSD1, LOL1 is constitutively expressed in all tissues. Its expression is unaltered in lsd1 null mutant plants grown under permissive conditions.

### Laboratory Example 2

### Analysis of LOL1 Transcription in Wild-Type and Mutant Plants

A T-DNA insertion mutant allele, lol1-1, was identified in which LOL1 transcripts can no longer be detected by mRNA blot analysis. The lol1-1 mutant was crossed to lsd1 and double mutants were identified using the polymerase chain reaction (PCR). Different primer sets were used to detect the lsd1 mutation and the T-DNA insertion. To detect the lsd1 mutation, the primers were 5′ACCTAACAAAAAGAAAAGTGTGTGAGG-3′ (SEQ ID NO: 13), 5′-ATAATAACCCCTACTAGCTCTAACAAG-3′ (SEQ ID NO: 14), and 5′-CTGCTACTTTCATCCAAAC-3′ (SEQ ID NO: 15). To detect the T-DNA insertion, the primers were 5′-TGAGTTATGAGCAATATAGAGGAA-3′ (SEQ ID NO: 16) and 5′-CATTTTATAATAACGCTGCGGACATCTAC-3′ (SEQ ID NO: 17).

### Laboratory Example 3

### Generation of Transgenic Arabidopsis Expressing Different Levels of LOL1

Transgenic Arabidopsis lines were generated that expressed either higher or lower levels than wild-type levels of LOL1 mRNA (LOL1-s and lol1-as, respectively). Transgenic lines were established in both the wild type Ws-0 and isogenic lsd1 null backgrounds according to the method of McDowell et al., 1998. Briefly, the LOL1 coding region was cloned in sense and antisense orientations into the binary vector pBAR1. LSD1/lsd1 heterozygotes were transformed using standard techniques, and at least 6 independent lines were identified per construct in the isogenic lsd1 and Ws-0 backgrounds. All experiments described in the Laboratory Examples were carried out with at least four independent lines per construct per genetic background.

RT-PCR was used to determine the relative mRNA levels of LOL1 in the various antisense transgenic lines using primers specific for the LOL1 gene. These primers were 5′-CGAAACGCGATTCTACAATTAGTC-3′ (SEQ ID NO: 18) and 5′-ATTCACTCCAAGAAGAATTGCAAT-3′ (SEQ ID NO: 19). The nucleotide mix with which the PCR reaction was performed contained a small amount of α-³²P dATP, which allowed the products of the PCR reaction to be analyzed using a phosphoimager device (such as are available from Amersham Biosciences of Sunnyvale, Calif., United States of America) after they were separated on a 1.5% agarose gel. For the sense lines, RNA was analyzed with standard Northern blotting and hybridization methods using the LOL1 coding region as a radiolabelled probe, followed by analysis using a phosphoimager device. These techniques demonstrated that LOL1 mRNA levels in the lol1-as lines were reduced to about 25-60% of wild type, and over-expression in LOL1-s lines resulted in ˜2-3 fold increase in LOL1 transcript amount.

### Laboratory Example 4

### LOL1 Function is Required for lsd1 Rcd

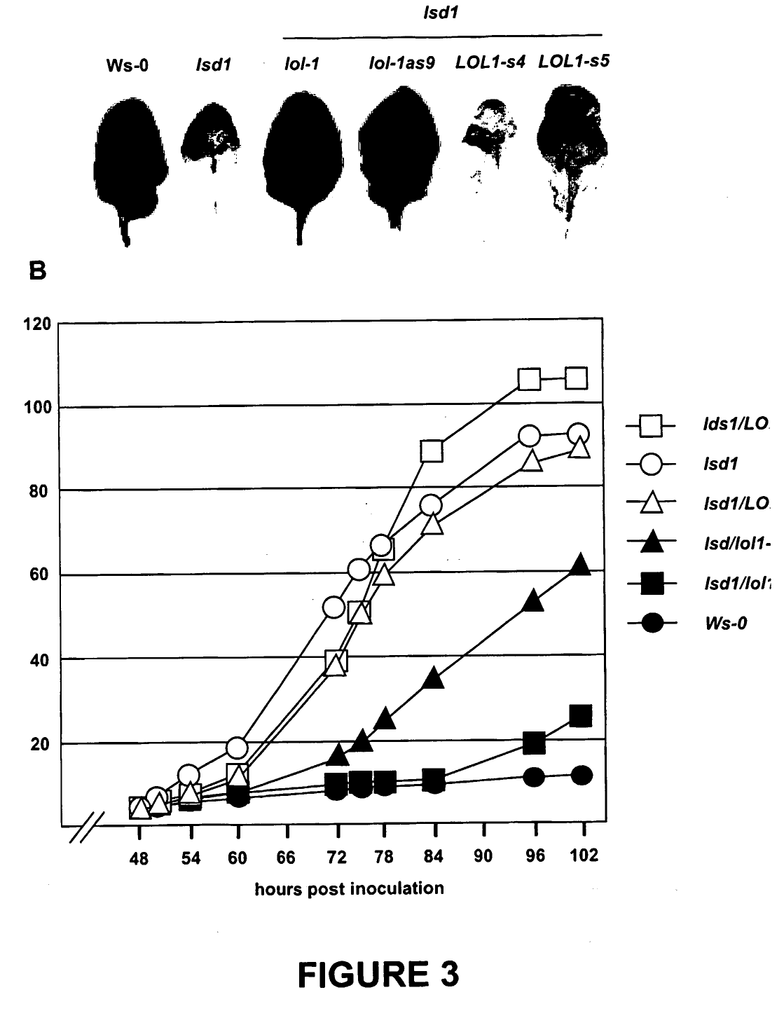

In order to address whether manipulation of LOL1 levels altered either the rcd induced in an lsd1 background or the wild type response to pathogens, plants were treated with benzo(1,2,3)-thiadiazole-7-carbothioic acid S-methyl ester (BTH). BTH induces rcd in lsd1 plants (Görlach et al., (1996) Plant Cell 8:629-643). Briefly, four-week-old plants from the lsd1/lol1-as and lsd1/LOL1-s lines were sprayed with 300 μM BTH and monitored for rcd. By 7 days post-BTH spray, rcd devastated lsd1 and lsd1/LOL1-s leaves; the tissue was collapsed and completely dried. In contrast, lsd1/lol1-as lines were predominantly healthy and green. See FIG. 3A.

Cell death was examined by measuring conductivity as an indicator of membrane damage and cellular ion leakage as described in Baker et al. (Baker et al., (1993) Physiol. Molec. Plant Pathol. 43: 81-94) and Dellagi et al. (Dellagi et al., (1998) Mol. Plant-Microbe Interact. 11: 734-742). Briefly, at various stages after treatment, a 7 mm diameter leaf disk was removed using a cork borer, floated in distilled water for 45 minutes, and transferred to tubes containing 6 ml of distilled water. Conductivity was determined with an Orion conductivity meter (available from Thermo Orion of Beverly, Massachusetts, United States of America) at various time points. Mean and standard error were calculated from 4 disks per genotype, with 3 repetitions within an experiment, and each experiment repeated 4 times. Ws-0 tissue did not exhibit any significant cell death and thus no increase in conductivity was observed, while lsd1 mutant tissue reached a maximum conductivity at 96 hours after BTH treatment. Reduction of LOL1 transcript levels in the lsd1/lol1-as lines, or in lsd1/lol1-1 significantly reduced conductivity compared to either lsd1 or the lsd1/LOL1-s lines. See FIG. 3B.

### Laboratory Example 5

### Infection of Transgenic Arabidopsis with Botrytis cinerea

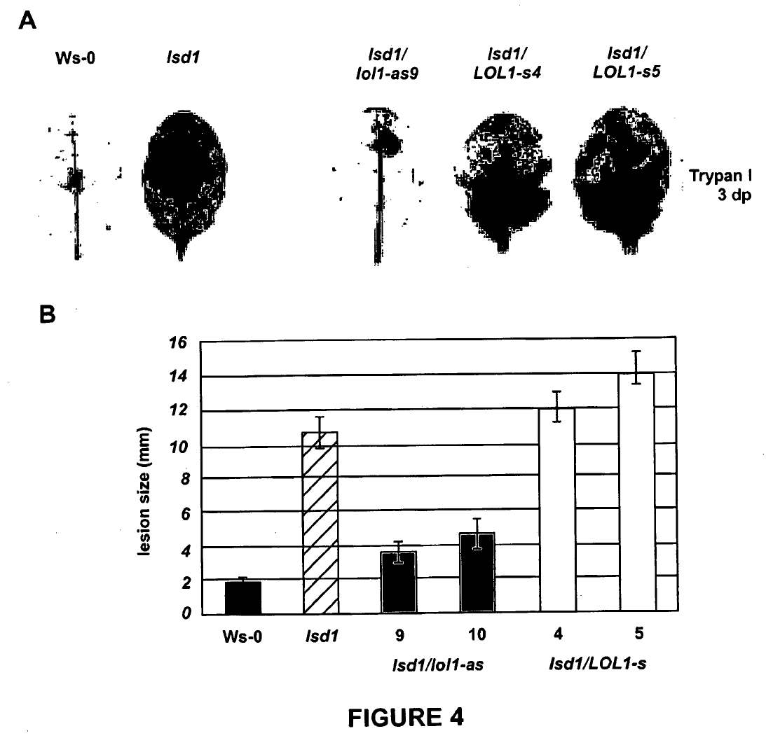

In order to assess the characteristics of rod in transgenic Arabidopsis plants that expressed different levels of LOL1, plants were infected with the necrotrophic pathogen Botrytis cinerea to activate rod. The lsd1 rod phenotype is accelerated following B. cinerea infection, presumably because this fungus produces Reactive Oxygen Intermediates (ROI) as part of its pathogenicity program (Govrin & Levine, (2000) Curr. Biol. 10: 751-757). 4-week-old plants were drop-inoculated with B. cinerea and lsd1 rcd was visualized by lactophenol trypan blue staining to identify dead cells (Keogh et al., (1980) Trans. Br. Mycol. Soc. 74: 329-333). The particular isolate of B. cinerea used is weakly pathogenic on wild-type (i.e., LSD1-positive) Arabidopsis, in which staining is limited to the site of infection. In contrast, lsd1 leaves were killed by rcd and fungal proliferation. The lsd1/lol-as lines, on the other hand, exhibited significantly reduced rod, while the lsd1/LOL-s lines were at least as susceptible as lsd1. Lesion size was measured on several leaves per genotype. Reduction of LOL1 function clearly attenuated rod in the lsd1 background, whereas over-expression of LOL1 mRNA moderately enhanced rcd. See FIGS. 4A and 4B.

### Laboratory Example 6

### LOL1 is an Enhancer of HR

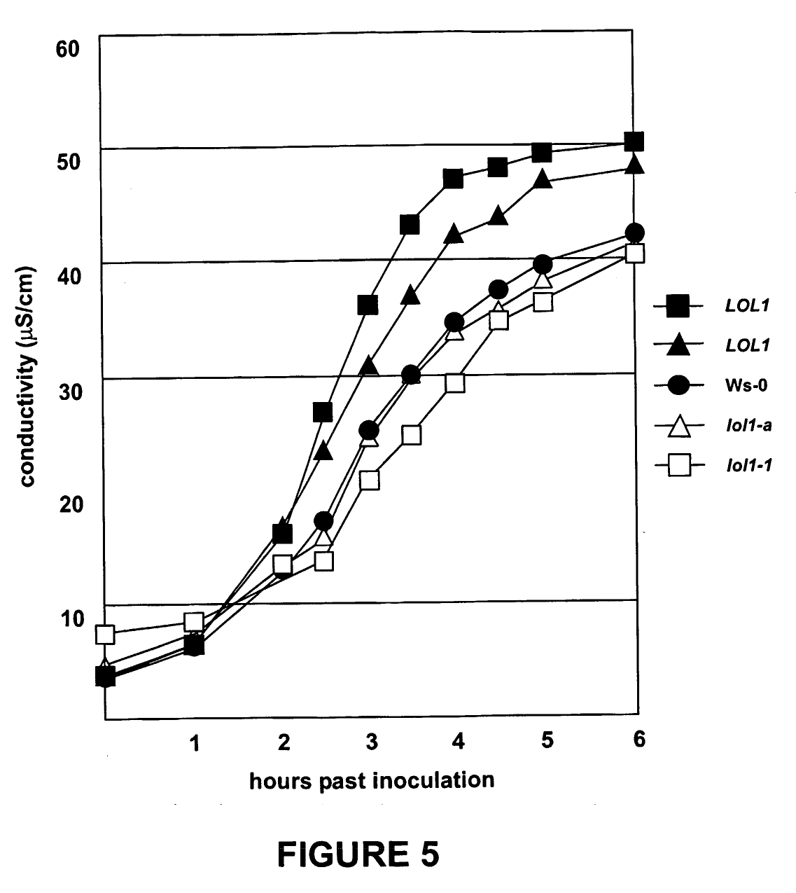

The LOL1 function observed in the aforementioned Examples was revealed in the context of an already poised ectopic cell death phenotype, namely, an lsd1 mutant genotype. In order to determine whether mis-regulation of LOL1 could also influence HR in a wild type background, Pst DC3000(avrRpm1) was used to trigger HR through the action of the RPM1 disease resistance gene (Grant et al., (1995) Science 269: 843-846) in the Ws-0, LOL1-s and lol1-as backgrounds. Briefly, 4-week-old plants were infiltrated with a 10 mM MgCl₂ solution containing Pst DC3000(avrRpm1) at a concentration of 5×10⁷ cfu/ml. Immediately thereafter, leaf disks were removed and processed as in Example 4. The onset of HR was quantified using conductivity measurements as in Example 4. The onset of HR using this assay was at 2 hours post-inoculation (hpi), and maximum conductivity was reached at 6 hpi. Thus, this assay correlated with the observed onset of RPM1-dependent HR where tissue collapse is visible at ˜3 hpi and full tissue collapse is evident by 6 hpi as reported by Dangl et al. (Danql et al., (1992) Plant Cell 4: 1359-1369). The LOL1-s lines reached the maximum wild type conductivity level by 3.5 hpi, and achieved plateau levels 20% higher than wild type. In this assay, therefore, the time course of cell collapse was accelerated in the LOL1-s lines. RPM1-mediated HR in the lol1-as lines was essentially wild type. See FIG. 5.

### Laboratory Example 7

### LOL1 Over-Expression Leads to Enhanced Pathogen Resistance

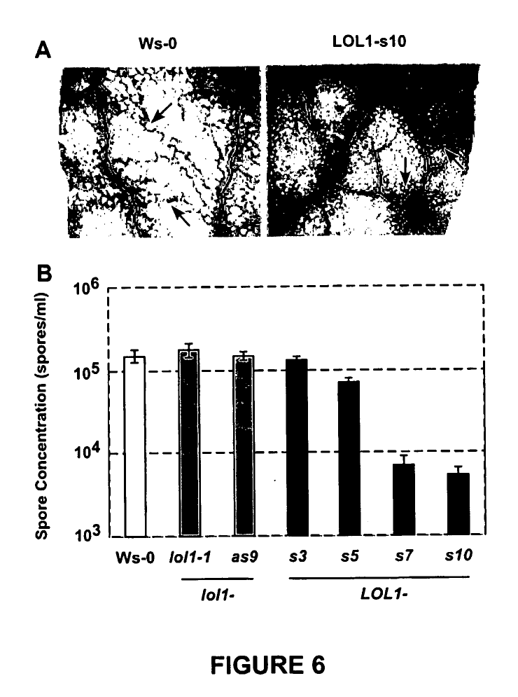

Since LOL1 has been shown to influence rcd, LOL-1s lines were tested for enhanced resistance to virulent pathogens by virtue of enhanced HR. 4-week-old plants were sprayed with the virulent P. parasitica isolate Emco5 at 1×10⁴ spores/ml. Inoculated leaves were stained with lactophenol trypan blue at 5 days post inoculation (dpi). Susceptibility was quantified by determining the number of spores produced on each genotype. All the leaves from one plant were collected, their weight was measured, and the leaves were transferred to a 15 ml tube. 500 ml distilled water was added per 100 mg of fresh weight. After vigorous vortexing, spores were counted in a hemocytometer. Mean and standard error were calculated from 5 repetitions. The experiment was repeated 3 times with similar results. Inoculation of LOL1-s lines with the virulent oomycete pathogen P. parasitica Emco5 was shown to lead to enhanced resistance. Pathogen sporulation on two LOL-s lines was reduced to 5% of wild type and other LOL1-s lines exhibited lesser decreases in susceptibility. Conversely, lol1-as lines showed slight increases in susceptibility. See FIGS. 6A and 6B.

### References

The references listed below as well as all references cited in the specification are incorporated herein by reference to the extent that they supplement, explain, provide a background for or teach methodology, techniques and/or compositions employed herein.
Adelman et al., (1983) DNA 2:183
Altschul et al., (1990) J. Mol. Bio. 215: 403-10
Ausubel et al., (1992) Current Protocols in Molecular Biology John Wylie and Sons, Inc. New York
Baker et al., (1993) Physiol. Molec. Plant Pathol. 43: 81-94
Bendahmane et al., (1999) Plant Cell 11: 781-791
Bevan et al., (1983) Nature 304:184-187
Bi et al., (1995) Plant J. 8: 235-245
Binet et al., (1991) Plant Science 79: 87-94
Biochinger & Diggelmann, Mol Cell Biol4: 2929-2931
Brown et al., (1985) FEBS Lett. 186:271-274
Callis et al., (1987) Genes Develop. 1: 1183-1200
Cameron et al., (1994) Plant J. 5: 715-725
Charron et al., (1999) Mol. Cell Biol. 19: 4355-4365;
Chibbar et al., (1993) Plant Cell Rep. 12: 506-509
Christensen et al., (1989) Plant Molec. Biol. 12: 619-632
Christou et al., (1991) Biotechnology 9: 957-962
Comai et al., (1988) J. Biol. Chem. 263: 15104-15109
Conner et al., (1983) Proc. Natl. Acad. Sci. U.S.A. 80:278
Creighton, (1983) Proteins: Structures and Molecular Principles, W. H. Freeman & Co., New York
Crop Breeding, Wood (Ed.), American Society of Agronomy, Madison, Wis. (1983)
Dangl & Jones, (2001) Nature 411: 826-833;
Dangl et al., (1992) Plant Cell 4: 1359-1369
Dangl et al., in Biochemistry and Molecular Biology of Plants (Buchanan et al., eds.) (ASPP Press, Rockville, 2000) pp. 1044-1100;
Datta et al., (1990) Biotechnology 8: 736-740
de Framond, (1991) FEBS 290: 103-106
Delaney et al., (1994) Science 266, 1247-1250
Delaney et al., (1995) Proc. Natl. Acad. Sci. USA 92, 6602-6606
Delaney, (1997) Plant Phys. 113: 5-12
Dellagi et al., (1998) Mol. Plant-Microbe Interact. 11: 734-742
Dempsey & Klessig, (1995) Bulletin de L'Institut Pasteur 93: 167-186
Deveraux & Reed, (1999) Genes Dev. 13: 239-252;
Dietrich et al., (1994) Cell 77: 565-577
Dietrich et al., (1997) Cell 88: 685-694
Edelmann et al., (eds.) Methods in Chloroplast Molecular Biology, Elsevier pp. 1081-1091 (1982)
Feys & Parker, (2000) Trends Genet. 16: 449-455;
Firek et al., (1993) Plant Molec. Biol. 22: 129-142
Fromm et al., (1990) Biotechnology 8: 833-839
Gaffney et al., (1993) Science 261, 754-756
Gallie et al., (1987) Nucl. Acids Res. 15: 8693-8711
Glover, (ed.) (1985) DNA Cloning: A Practical Approach, MRL Press, Ltd., Oxford, U.K
Gordon-Kamm et al., (1990) Plant Cell 2: 603-618
Görlach et al., (1996) Plant Cell 8: 629-643
Gorman et al., (1996) Immunity 5: 241-252
Govrin & Levine, (2000) Curr. Biol. 10: 751-757
Grant et al., (1995) Science 269: 843-846
Greenberg & Ausubel, (1993) Plant J. 4: 327-342
Greenberg et al., (1994) Cell 77: 551-563
Gribskov et al., (1986) Nucl. Acids. Res. 14: 6745
Harlow & Lane, (1988) Antibodies: A Laboratory Manual Cold Spring Harbor Laboratory Press, Cold Spring Harbor, New York
Hofgen & Willmitzer, (1988) Nucl. Acids Res. 16: 9877
Hudspeth & Grula, (1989) Plant Molec Biol 12: 579-589
Hunt & Ryals, (1996) Crit. Rev. in Plant Sci. 15, 583-606
Jabs et al., (1996) Science 273: 1853-1856
Jones & Dangl, (1996) Trends Plant Science 1: 114-119;
Keogh et al., (1980) Trans. Br. Mycol. Soc. 74: 329-333
Klein et al., (1987) Nature 327: 70-73
Koehler & Ho, (1990) Plant Cell 2: 769-783
Korsmeyer, (1995) Trends Genet. 11: 101-105
Koziel et al., (1993) Biotechnology 11: 194-200
Kyte & Doolittle, (1982), J. Mol. Biol. 157: 105-132
Landgren et. al., (1988) Science 241: 1007
Landgren et. al., (1988) Science 242: 229-237
Lawton et al., (1996) Plant J. 10: 71-82
Logemann et al., (1989) Plant Cell 1: 151-158
Luckow and Schutz, (1987) Nucleic Acids Res 15: 5490
Maher et al., (1994) Proc. Natl. Acad. Sci. USA 91: 7802-7806
Mauch-Mani & Slusarenko, (1994) Mol. Plant-Microbe Interact. 7: 378-383
Mauch-Mani & Slusarenko, (1996) Plant Cell 8: 203-212
Mayo, The Theory of Plant Breeding, Second Edition, Clarendon Press, Oxford (1987)
McDowell & Dangl, (2000) Trends Biochem. Sci. 25: 79-82
McElroy et al., (1990) Plant Cell 2: 163-171
McElroy et al., (1991) Mol. Gen. Genet. 231: 150-160
Messing & Vierra, (1982) Gene 19: 259-268;
Miller et al., (1985) EMBO 4:1609-1614;
Miller, (1999) Trends Cell Biol. 9: 323-328;
Molkentin, (2000) J. Biol. Chem. 275: 38949-38952;
Morel & Dangl, (1997) Cell Death and Different. 19: 17-24
Murashiga & Skoog, (1962) Physiologia Plantarum 15: 473-497
Needleman et al., (1970) J. Mol. Biol. 48: 443
Pallas et al., (1996) Plant J. 10: 281-293
Paszkowski et al., (1984) EMBO J 3: 2717-2722
Peterhänsel et al., (1997) Plant Cell 9: 1397-1409
Pevny et al., (1991) Nature 349: 257-260
Potrykus et al., (1985) Mol. Gen. Genet. 199: 169-177
Reich et al., (1986) Biotechnology 4: 1001-1004
Rogers et al., (1985) Proc. Natl. Acad. Sci. USA 82: 6512-6516
Rohrmeier & Lehle, (1993) Plant Molec. Biol. 22: 783-792
Ryals et al., (1996) Plant Cell 8, 1809-1819
Saiki et al., (1985) Bio/Technology 3:1008-1012
Sambrook et al., (eds.) (1989) Molecular Cloning, Cold Spring Harbor Laboratory Press, Cold Spring Harbor, New York
Schwartz et al., (eds.), (1979), Atlas of Protein Sequence and Structure. National Biomedical Research Foundation, pp. 357-358
Shimamoto et al., (1989) Nature 338: 274-277;
Shinshi et al., (1990) Plant Molec. Biol. 14: 357-368
Shirasu & Schulze-Lefert, (2000) Plant Molec. Biol. 44: 371-385
Shulaev et al., (195) Plant Cell 7: 1691-1701
Silhavy et al., (1984) Experiments with Gene Fusions, Cold Spring Harbor Laboratory Press, Cold Spring Harbor, New York

Singh, Breeding for Resistance to Diseases and Insect Pests, Springer-Verlag, New York (1986)
Skuzeski et al., (1990) Plant Molec. Bio. 15: 65-79
Smith et al., (1981) Adv. Appl. Math. 2:482
Spencer et al., (1990) Theor. Appl. Genet 79: 625-631
Taylor et al., (1993) Plant Cell Rep. 12: 491-495
Tijssen, (1993) Laboratory Techniques in Biochemistry and Molecular Biology—Hybridization with Nucleic Acid Probes, part 1, chapter 2, Elsevier, New York
Uknes et al., (1992) Plant Cell 4: 645-656
Uknes et al., (1993) Mol. Plant-Microbe Interact. 6: 692-698
Uknes et al., (1993) Plant Cell 5: 159-169
Unger et al., (1989) Plant Molec. Biol. 13: 411-418
van den Broeck et al., (1985) Nature 313: 358-363
Vasil et al., (1992) Biotechnology 10: 667-674
Vasil et al., (1993) Biotechnology 11: 1553-1558
Verhagen et al., (2001) Genome Biol. 2
Vernooij et al., (1994) Plant Cell 6: 959-965
Vernooij et al., (1995) Mol. Plant-Microbe Interact. 8: 228-234
Walbot et al., in Genetic Engineering of Plants, (Kosuge & Meredith, eds.) Plenum Publishing Co., New York, 1983, vol. 3, pp. 431-442
Ward et al., (1991) Plant Cell 3: 1085-1094
Warner et al., (1993) Plant J. 3: 191-201
Wasmann et al., (1986) Mol. Gen. Genet. 205: 446-453
Weeks et al., (1993) Plant Physiol. 102: 1077-1084
Wellner, (1971) Anal. Chem. 43: 597
Welsh, Fundamentals of Plant Genetics and Breeding, John Wiley & Sons, New York (1981)
Wetmur & Davidson, (1968) J. Mol. Biol. 31: 349-70
Weymann et al., (1995) Plant Cell 7: 2013-2022
White et al., (1990) Nucl. Acids Res 18: 1062
Wricke & Weber, Quantitative Genetics and Selection Plant Breeding, Walter de Gruyter and Co., Berlin (1986)
Xu et al., (1993) Plant Molec. Biol. 22: 573-588
Zhang et al., (1988) Plant Cell Rep 7: 379-384
U.S. Pat. No. 4,196,265
U.S. Pat. No. 5,986,173
U.S. Pat. No. 4,554,101
U.S. Pat. No. 4,940,935
U.S. Pat. No. 4,945,050
U.S. Pat. No. 5,036,006
U.S. Pat. No. 5,100,792
U.S. Pat. No. 5,188,642
U.S. Pat. No. 5,260,203
U.S. Pat. No. 5,260,203
U.S. Pat. No. 5,591,616
U.S. Pat. No. 5,614,395
U.S. Pat. No. 5,614,395
U.S. Pat. No. 5,639,949
U.S. Pat. No. 5,792,904
U.S. Pat. No. 5,948,953
U.S. Pat. No. 5,952,546
U.S. Pat. No. 6,091,004
U.S. Pat. No. 6,031,153
U.S. Pat. No. RE 36,449
WO 93/07278
WO 93/21335
WO 94/00977
EP 0 292 435
EP 0 392 225

It will be understood that various details of the invention may be changed without departing from the scope of the invention. Furthermore, the foregoing description is for the purpose of illustration only, and not for the purpose of limitation the invention being defined by the claims.

1. An isolated and purified biologically active LOL1 polypeptide that acts as a positive regulator of programmed cell death in a plant.

2. The polypeptide of claim 1, comprising an amino acid sequence selected from the group consisting of SEQ ID NOs: 2 and 4-12, sequences having at least 85% identity with one of SEQ ID NOs: 2 and 4-12, and fragments thereof.

3. A chimeric polypeptide comprising an amino acid sequence selected from the group consisting of SEQ ID NOs: 2 and 4-12.

4. A polypeptide of claim 1 in a detectably labeled form.

5. An antibody that selectively recognizes a polypeptide of claim 1.

7. An isolated and purified nucleic acid sequence encoding a polypeptide that acts as a positive regulator of PCD in a plant.

8. The isolated and purified nucleic acid sequence of claim 7, wherein the nucleic acid sequence comprises a nucleic acid sequence selected from the group consisting of:
(a) SEQ ID NO: 1;
(b) a sequence encoding a polypeptide comprising a an amino acid sequence selected from the group consisting of SEQ ID NOs: 2 and 4-12; and
(c) a nucleic acid sequence capable of hybridizing under stringent conditions to a nucleic acid sequence according to (a) or (b).

9. The nucleic acid sequence of claim 7, wherein the nucleic acid sequence is a DNA sequence.

10. A chimeric gene comprising a nucleic acid sequence of claim 7 or 8 operatively linked to a promoter.

11. A recombinant vector comprising the chimeric gene of claim 10.

12. A host cell stably transformed with the recombinant vector of claim 11.

13. A plant stably transformed with the recombinant vector of claim 11.

14. The transgenic plant of claim 14, wherein the nucleic acid sequence is present in the genome in a copy number effective to confer expression in the plant of a LOL1 polypeptide that acts as a positive regulator of programmed cell death in a plant.

15. A seed derived from a transgenic plant of claim 13.

16. Progeny derived from a transgenic plant of claim 13.

17. A part of a transgenic plant of claim 13.

18. The transgenic plant of claim 13, wherein the plant is selected from the group consisting of Arabidopsis, rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

19. A method of detecting a nucleic acid molecule that encodes a LOL1 polypeptide that acts as a positive regulator of programmed cell death in a plant in a biological sample containing nucleic acid material, the method comprising:
(a) hybridizing a nucleic acid sequence of claim 7 or 8 to nucleic acid material of a biological sample under stringent hybridization conditions, to form a hybridization duplex; and
(b) detecting the hybridization duplex.

20. The method of claim 19, wherein the detected nucleic acid molecule further comprises a chromosome.

21. A method of identifying positive regulation of programmed cell death in a plant, the method comprising:
(a) contacting a query nucleic acid sequence derived from a plant with a probe comprising a nucleic acid sequence of claim 7 or 8; and
(b) detecting the formation of a hybridized structure comprising the probe and the query nucleic acid sequence, the presence of a hybridized structure being indicative of positive regulation of programmed cell death in the plant.

22. The method of claim 21, wherein the probe comprises a nucleotide sequence complementary to a nucleic acid sequence of claim 7 or 8.

23. The method of claim 21, wherein the plant is selected from the group consisting of Arabidopsis, rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

24. A method of increasing LOL1 gene expression in a plant comprising transforming the plant with the recombinant vector of claim 11.

25. A method of enhancing disease resistance in a plant comprising transforming a plant with the recombinant vector of claim 11.

26. The method of either of claims 24 or 25, wherein the recombinant vector is expressed in the plant at higher levels than in a wild type plant.

27. The method of of either of claims 24 or 25, wherein the plant is selected from the group consisting of Arabidopsis, rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane

28. An assay kit for detecting the presence of a LOL1 polypeptide that acts as a positive regulator of programmed cell death in a plant, the assay kit comprising a first container containing a nucleic acid probe comprising a sequence of ten or more contiguous nucleotide bases corresponding to a fragment of a nucleic acid sequence of claim 7 or 8.

29. The kit of claim 28, further comprising a second container containing a detectable moiety.
